(19) United States
(12) Patent Application Publication
(10) Pub. No.: US 2024/0299384 A1
(43) Pub. Date: Sep. 12, 2024

(54) METHODS FOR TREATING PULMONARY HYPERTENSION IN PATIENTS WITH LEFT VENTRICULAR ASSIST DEVICE IMPLANTATION

(71) Applicant: ACTELION PHARMACEUTICALS LTD, Allschwil (CH)

(72) Inventors: Mark ROCCO, South San Francisco, CA (US); Mona SELEJ, South San Francisco, CA (US); Carol ZHAO, South San Francisco, CA (US)

(21) Appl. No.: 18/547,959

(22) PCT Filed: Feb. 25, 2022
(86) PCT No.: PCT/EP2022/054742

§ 371 Date: Aug. 25, 2023

Related U.S. Application Data
(60) Provisional application No. 63/154,348, filed on Feb. 26, 2021.

(51) Int. Cl. A61K 31/506; A61K 9/00; A61K 9/28; A61M 60/90; A61P 9/12

(52) U.S. Cl. CPC A61K 31/506; A61K 9/0053; A61K 9/28; A61M 60/90; A61P 9/12

(57) ABSTRACT

The present disclosure provides methods for treating pulmonary hypertension in a patient with left ventricular assist device (LVAD) implantation and methods of improving cardiac transplant eligibility in a patient with LVAD implantation. The methods include administering to a patient in need thereof a therapeutically effective amount of macitentan or aprocitentan.

### TECHNICAL FIELD

This invention relates to methods for treating pulmonary hypertension in patients with left ventricular assist device implantation.

### BACKGROUND

End-stage heart failure (HF) is a debilitating condition for which heart transplant offers the best treatment option, but the need for donor hearts greatly exceeds supply. Since their first approval, left ventricular assist devices (LVADs) have become an increasingly frequent treatment option for patients with end-stage HF. While LVADs improve left-sided hemodynamic variables, right ventricular failure (RVF) occurring after the implantation of LVADs is a major cause of morbidity and mortality. Persistently elevated pulmonary vascular resistance (PVR) is associated with RVF after LVAD implantation.

The management of PH post-LVAD implantation starts with measures to prevent RVF in the preoperative period. Post-operatively, the management of PH complicated with RVF in these patients includes the use of pulmonary vasodilators, changing the pump speed under echocardiography guidance, and, if refractory RVF, consideration of a right ventricular assist device (RVAD) implantation. To date, no targeted PAH therapy has demonstrated efficacy and safety from a randomized controlled trial in this patient population.

What is needed are therapies that manage PH in patients with PH post-LVAD implantation.

### SUMMARY

In certain embodiments, the disclosure provides methods for treating pulmonary hypertension in a patient with left ventricular assist device (LVAD) implantation, comprising administering to a patient in need thereof a therapeutically effective amount of macitentan.

In other embodiments, the disclosure provides methods for treating pulmonary hypertension in a patient with LVAD implantation, comprising administering to a patient in need thereof a therapeutically effective amount of aprocitentan.

In further embodiments, the disclosure provides methods of improving cardiac transplant eligibility in a patient with LVAD implantation, comprising administering to a patient in need thereof a therapeutically effective amount of macitentan.

In yet other embodiments, the disclosure provides methods of improving cardiac transplant eligibility in a patient with LVAD implantation, comprising administering to a patient in need thereof a therapeutically effective amount of aprocitentan.

In still further embodiments, the disclosure provides macitentan for treating pulmonary hypertension in a patient with left ventricular assist device (LVAD) implantation, comprising administering an amount of macitentan, wherein the LVAD implantation is within about 90 days prior to administering the macitentan.

In other embodiments, the disclosure provides aprocitentan for treating pulmonary hypertension in a patient with left ventricular assist device (LVAD) implantation, comprising administering an amount of aprocitentan, wherein the LVAD implantation is within about 90 days prior to administering the aprocitentan.

In further embodiments, the disclosure provides macitentan for improving cardiac transplant eligibility in a patient with left ventricular assist device (LVAD) implantation, comprising administering an amount of macitentan, wherein the LVAD implantation is within 90 days prior to administering the macitentan.

In yet other embodiments, the disclosure provides aprocitentan for improving cardiac transplant eligibility in a patient with left ventricular assist device (LVAD) implantation, comprising administering an amount of aprocitentan, wherein the LVAD implantation is within 90 days prior to administering the aprocitentan.

### BRIEF DESCRIPTION OF THE DRAWINGS

FIG. 1 is a schematic of the treatment described in Example 1. In this figure, ^{a}LVAD implant must occur within 90 days (inclusive) prior to the date of randomization and randomization must occur within 14 days after qualifying RHC; ^{b}Patients with documented severe obstructive lung disease, moderate to severe restrictive lung disease, or pulmonary veno-occlusive disease are excluded; ^{c}All secondary and exploratory endpoints are calculated as change from baseline to week 12.

FIG. 2 is a schematic summarizing the disposition of patients of Example 1.

FIG. 3 is a scatter plot showing the proportion of patients achieving a PVR<3 WU.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

### Methods

The methods described herein are directed to treating pulmonary hypertension (PH) in patients who have an implanted left ventricular assist device (LVAD). Such patients are not candidates for cardiac transplant due to increased risk of post-transplantation right ventricular failure and mortality. In most of post-LVAD patients who continue to have PH, medication is often prescribed as a standard of care. However, not all post-LVAD patients require additional PH treatments and may be unnecessarily treated with additional medications or treated with medications that have not demonstrated a consistent effect. Thus, in certain embodiments, the methods described herein aim to prevent administration of unnecessary PH medications to patients and, instead, treat only those patients requiring post-LVAD PH medications, e.g., those post-LVAD patients with persistent PH.

The methods described herein also improve cardiac transplant eligibility in patients with LVAD implantation, particularly, for example, in patients in which cardiac transplant is contraindicated due to elevated PVR. The methods described herein can result in a patient moving from not being considered a candidate for orthoptic heart transplant to becoming a candidate who is listed for heart transplant following treatment with macitentan or aprocitentan. For patients with advanced heart failure who are already on the heart transplant waitlist, developing a PVR>3 WU would result in their removal from the transplant list due to the risk of post-transplantation RVF and death. These methods could keep their PVR in the favorable range (<3 WU) for transplant eligibility.

As used herein and in the art, the terms “pulmonary hypertension” and “PH” are interchangeable and define a general condition where a patient has high blood pressure in the arteries of the lungs. PH occurs when the very small arteries throughout the lungs narrow in diameter, which increases the resistance to blood flow through the lungs. PH is classified into subgroups including (i) pulmonary arterial hypertension (PAH), (ii) PH due to left heart disease, (iii) PH due to lung disease, (iv) PH due to chronic blood clots (CTEPH), or (v) PH due to miscellaneous diseases. In some embodiments, the methods desirably treat PAH.

The terms “pulmonary arterial hypertension” and “PAH” are interchangeable and define pulmonary hypertension that is chronic whereby the walls of the arteries of the lungs tighten and stiffen. In some embodiments, the underlying cause of the narrowing is not known, i.e., idiopathic pulmonary hypertension (iPAH). PAH also is classified into subgroups including (i) familial, or heritable PAH, (ii) PAH caused by drugs or toxins, (iii) PAH associated with other conditions such as connective tissue diseases (scleroderma or lupus), congenital heart problems, high blood pressure in the liver, HIV, and infections (schistosomiasis), (iv) PAH caused by rare blood conditions (e.g. pulmonary capillary hemangiomatosis), or (v) PAH in babies (persistent pulmonary hypertension of the newborn). Severity of PAH in a patient is generally evaluated by a classification system, i.e., the World Health Organization (WHO) class system. See, Table 1.

TABLE 1
WHO Functional Class System for PAH Patients

| Class | Patient's Symptoms |
|---|---|
| I | No limitations on physical activity. |
| | Ordinary physical activity does not cause undue |
| | dyspnea or fatigue, chest pain, or near syncope. |
| II | Slight limitation of physical activity. |
| | Comfortable at rest. |
| | Ordinary physical activity causes undue dyspnea |
| | or fatigue, chest pain, or near syncope. |
| III | Marked limitation of physical activity. |
| | Comfortable at rest. |
| | Less than ordinary activity causes undue dyspnea |
| | or fatigue, chest pain or near syncope. |
| IV | Inability to carry out any physical activity |
| | without symptoms. |
| | Manifest signs of right heart failure. |
| | Dyspnea and/or fatigue may be present at rest. |
| | Discomfort is increased by any physical activity. |

The methods, thus, may provide a better quality of life or overall survival for post-LVAD patients. Patients eligible for the treatment methods described herein may be selected by one skilled in the art, i.e., the attending physician. In some embodiments, the patient does not have severe hepatic impairment, e.g., Child-Pugh class C liver disease. As known in the art, the “Pugh-Child score” is determined by scoring five clinical measures of liver disease and the totals of those scores place a patient in either class A, B, or C. See, Table 2.

TABLE 2

| Class | A | B | C |
|---|---|---|---|
| Points | 5-6 | 7-9 | 10-15 |
| Severity of Liver Disease | Least | Moderate | Most |
| Survival Rate | 95% | 75% | 50% |

In other embodiments, the patient does not have a severe obstructive lung disease defined as an FEV₁/FVC<about 0.7 associated with a FEV₁<about 50% of predicted value after bronchodilator administration. In further embodiments, the patient does not have moderate to severe restrictive lung disease defined as a total lung capacity <about 60% of predicted value. In yet other embodiments, the patient does not have pulmonary veno-occlusive disease. In still further embodiments, the patient is not undergoing dialysis. In other embodiments, the patient's hemoglobin is >about 8.5 g/dL. In further embodiments, the patient's AST or ALT is <about 3 times the upper limit of normal. In still other embodiments, the patient's doppler mean blood pressure is greater than about 65 mmHg. In yet further embodiments, the patient's GFR is >about 30 mL/min.

Eligible patents may also be on a cardiac transplant waiting list. In some embodiments, the methods described herein result in the patient moving up on the waitlist. In other embodiment, the methods result in the patient remaining on the waitlist. Alternatively, the methods result in a patient being added to a cardiac transplant waiting list.

The term “LVAD” as used herein refers to a left ventricular assist device. The LVAD is a battery-operated, mechanical pump, which helps the left ventricle (main pumping chamber of the heart) pump blood to the rest of the body. The LVAD used herein includes a number of components including (i) a surgically implanted pump, (ii) an inflow cannula, (iii) outflow graft, (iv) batteries with a life span up to 12 hours, (v) percutaneous driveline and (vi) system controller. In some embodiments, the pump works in parallel with the heart via the inflow cannula to the left ventricle and an outflow graft to the ascending aorta. In other embodiments, the LVAD is a continuous-flow axial pump, centrifugal pump, or mixed design pumps where the pump is axial, but the blood exits perpendicular to the inflow as in the centrifugal pump. LVAD instruments are known in the art and include the HVAD® (Medtronic), HeartMate II™ (Abbott), and HeartMate 3™ (Abbott), without limitation.

LVAD implantation is desirably within about 90 days prior to initiating treatment with macitentan or aprocitentan. In some embodiments, LVAD implantation is within about 90 days, about 80 days, about 75 days, about 70 days, about 65 days, about 60 days, about 55 days, about 50 days, about 45 days, about 40 days, about 35 days, about 30 days, about 25 days, about 20 days, about 15 days, about 10 days, or about 10 days prior to initiating treatment with macitentan or aprocitentan. In further embodiments, LVAD implantation is within about 5 to about 90 days, about 5 to about 80 days, about 5 to about 70 days, about 5 to about 60 days, about 5 to about 50 days, about 5 to about 40 days, about 5 to about 30 days, about 5 to about 20 days, about 5 to about 10 days, about 10 to about 90 days, about 10 to about 80 days, about 10 to about 70 days, about 10 to about 60 days, about 10 to about 50 days, about 10 to about 40 days, about 10 to about 30 days, about 10 to about 20 days, about 20 to about 90 days, about 20 to about 80 days, about 20 to about 70 days, about 20 to about 60 days, about 20 to about 50 days, about 20 to about 40 days, about 20 to about 30 days, about 30 to about 30 to about 90 days, about 30 to about 80 days, about 30 to about 70 days, about 30 to about 60 days, about 30 to about 50 days, about 30 to about 40 days, about 40 to about 90 days, about 40 to about 80 days, about 40 to about 70 days, about 40 to about 60 days, about 40 to about 50 days, about 45 to about 90 days, about 45 to about 80 days, about 45 to about 70 days, about 45 to about 60 days, about 50 to about 90 days, about 50 to about 80 days, about 50 to about 70 days, about 50 to about 60 days, about 60 to about 90 days, about 60 to about 80 days, about 60 to about 70 days, about 70 to about 90 days, about 70 to about 80 days, or about 80 to about 90 days prior to initiating treatment with macitentan or aprocitentan. In other embodiments, LVAD implantation is within about 90 days prior to initiating treatment with macitentan or aprocitentan. In further embodiments, LVAD implantation is within greater than 45 days to about 90 days prior to initiating treatment with macitentan or aprocitentan.

The methods include administering to a patient in need thereof a therapeutically effective amount of a macitentan or aprocitentan. After LVAD implantation and prior to initiating treatment with macitentan or aprocitentan, the patient has a mean pulmonary arterial pressure (mPAP) of about 25 mmHg or greater at rest. The term “at rest” as used herein refers to a period of time whereby the patient is inactive, e.g., sitting in absence of motion. In some embodiments, the patient has a mPAP of about 30 mmHg, about 35 mmHg, about 40 mmHg, about 45 mmHg, about 50 mmHg, about 55 mmHg, about 60 mmHg, about 65 mmHg, about 70 mmHg, about 75 mmHg, about 80 mmHg, about 85 mmHg, about 90 mmHg, or about 95 mmHg, or about 100 mmHg or greater. In other embodiments, the patient has a mPAP of about 30 mmHg or greater. In further embodiments, the patient has a mPAP of about 40 mmHg or greater.

The patient may also have a pulmonary arterial wedge pressure (PAWP) of about 18 mmHg or less after LVAD implantation and prior to initiating treatment with macitentan or aprocitentan. In some embodiments, the patient's PAWP is about 18 mmHg, about 17 mmHg, about 16 mmHg, about 15 mmHg, about 14 mmHg, about 13 mmHg, about 12 mmHg, about 11 mmHg, about 10 mmHg, about 9 mmHg, about 8 mmHg, about 7 mmHg, about 6 mmHg, or about 5 mmHg or less. In further embodiments, the patient's PAWP is about 17 mmHg or less. In yet other embodiments, the patient's PAWP is about 15 mmHg or less. In still further embodiments, the patient's PAWP is about 12 mmHg or less. In other embodiments, the patient's PAWP is about 10 mmHg or less.

The patient may further have a pulmonary vascular resistance (PVR) of greater than about 3 WU (Wood Units) after LVAD implantation and prior to initiating treatment with macitentan or aprocitentan. In certain embodiments, the patient has a PVR of about 4 WU, about 5 WU, about 6 WU, about 6.5 WU, about 7 WU, about 8 WU, or about 9 WU after LVAD implantation and prior to initiating treatment with macitentan or aprocitentan. In other embodiments, the patient has a PVR of greater than about 3 to about 7 WU, greater than about 3 to about 6.5 WU, greater than about 3 to about 6 WU, greater than about 3 to about 5 WU, greater than about 3 to about 4 WU, about 4 to about 7 WU, about 4 to about 6 WU, about 4 to about 5 WU, about 5 to about 7 WU, about 5 to about 6 WU, or about 6 to about 7 WU.

The patient may also a mPAP of about 25 mmHg or greater at rest and a PAWP of about 18 mmHg or less after LVAD implantation and prior to initiating treatment with macitentan or aprocitentan. In further embodiments, the patient has a mPAP of about 25 mmHg or greater at rest and a PVR of greater than about 3 WU after LVAD implantation and prior to initiating treatment with macitentan or aprocitentan. In yet other embodiments, the patient has a PAWP of about 18 mmHg or less and a mPAP of about 25 mmHg or greater at rest and a PVR of greater than about 3 WU after LVAD implantation and prior to initiating treatment with macitentan or aprocitentan. In still further embodiments, the patient has a mPAP of about 25 mmHg or greater at rest, a PAWP of about 18 mmHg or less, and a PVR of greater than about 3 WU after LVAD implantation and prior to initiating treatment with macitentan or aprocitentan.

The patient also may have a transpulmonary gradient (TPG) of greater than about 12 mmHg after LVAD implantation and prior to initiating treatment with macitentan or aprocitentan. In some embodiments, the patient has a TPG of greater than about 15 mmHg, about 20 mmHg, about 25 mmHg, about 30 mmHg, about 35 mmHg, about 40 mmHg, about 45 mmHg, about 50 mmHg, about 55 mmHg, about 60 mmHg, about 65 mmHg, or about 70 mmHg or greater after LVAD implantation and prior to initiating treatment with macitentan or aprocitentan. In further embodiments, the patient has a TPG of about 12 to about 60 mmHg, about 12 to about 55 mmHg, about 12 to about 50 mmHg, about 12 to about 45 mmHg, about 12 to about 40 mmHg, about 12 to about 35 mmHg, about 12 to about 30 mmHg, about 12 to about 25 mmHg, about 12 to about 20 mmHg, about 15 to about 60 mmHg, about 15 to about 55 mmHg, about 15 to about 50 mmHg, about 15 to about 45 mmHg, about 15 to about 40 mmHg, about 15 to about 35 mmHg, about 15 to about 30 mmHg, about 15 to about 25 mmHg, about 20 to about 60 mmHg, about 20 to about 55 mmHg, about 20 to about 50 mmHg, about 20 to about 45 mmHg, about 20 to about 40 mmHg, about 20 to about 35 mmHg, about 20 to about 30 mmHg, about 25 to about 60 mmHg, about 25 to about 55 mmHg, about 25 to about 50 mmHg, about 25 to about 45 mmHg, about 25 to about 40 mmHg, about 25 to about 35 mmHg, about 30 to about 60 mmHg, about 30 to about 55 mmHg, about 30 to about 50 mmHg, about 30 to about 45 mmHg, about 30 to about 40 mmHg, about 35 to about 60 mmHg, about 35 to about 55 mmHg, about 35 to about 50 mmHg, about 35 to about 45 mmHg, about 40 to about 60 mmHg, about 40 to about 55 mmHg, about 40 to about 50 mmHg, about 45 to about 60 mmHg, about 45 to about 55 mmHg, about 50 to about 60 mmHg, or about 55 to about 60 mmHg after LVAD implantation and prior to initiating treatment with macitentan or aprocitentan. In other embodiments, the patient has a TPG of about 12 to about 45 mmHg after LVAD implantation and prior to initiating treatment with macitentan or aprocitentan.

Patients may further have a pulmonary arterial wedge pressure (PAWP) of about 18 mmHg or less after LVAD implantation and prior to initiating treatment with macitentan or aprocitentan. In some embodiments, patients may have a PAWP of about 17 mmHg, about 16 mmHg, about 15 mmHg, about 14 mmHg, about 13 mmHg, about 12 mmHg, about 11 mmHg, about 10 mmHg, about 9 mmHg, about 8 mmHg, about 7 mmHg, about 6 mmHg, about 5 mmHg, about 4 mmHg, about 3 mmHg, about 2 mmHg, or about 1 mmHg or less after LVAD implantation and prior to initiating treatment with macitentan or aprocitentan. In other embodiments, patients may have a PAWP of about 1 to about 15 mmHg, about 1 to about 10 mmHg, about 1 to about 5 mmHg, about 5 to about 15 mmHg, about 5 to about 10 mmHg, about 10 to about 15 mmHg after LVAD implantation and prior to initiating treatment with macitentan or aprocitentan.

The methods are desirably effective in reducing the patient's PVR, e.g., to a level that is in the normal range. For example, the methods are effective in reducing the patient's PVR to below about 3 WU. In other embodiments, the methods are effective in reducing the patient's TPG. For example, the methods are effective in reducing the patient's TPG to below about 12 mmHg. In further embodiments, the methods are effective in reducing the patient's PVR and TPG. For example, the methods are effective in reducing the patient's PVR to below about 3 WU and reducing the patient's TPG to below about 12 mmHg.

Typically, after implantation of the LVAD, patients meet baseline hemodynamic criteria of PH via right heart catheterization (RHC) during a last measurement prior to the first dose of drug. In particular embodiments, the patient has a mean pulmonary arterial pressure (mPAP) of ≥25 mmHg at rest, a pulmonary arterial wedge pressure (PAWP)≤18 mmHg, and a pulmonary vascular resistance (PVR)>3 WU (Wood Units) after LVAD implantation and prior to initiating treatment with macitentan or aprocitentan. More particularly, the patient has a mean pulmonary arterial pressure (mPAP) of ≥25 mmHg at rest, a pulmonary arterial wedge pressure (PAWP)≤18 mmHg, and a pulmonary vascular resistance (PVR)>3 WU (Wood Units) within about 14 days prior to initiating the treatment with macitentan or aprocitentan. Establishing such timing may, for example, avoid administration of unnecessary PH medications to patients and, instead, treat those patients requiring post-LVAD PH medications.

The methods may also be effective in reducing the patient's PVR by at least about 10% relative to a patient population at the same level of disease diagnosis that is not receiving treatment with macitentan or aprocitentan. The term “patient population” as used herein refers a number of patients to provide statistically reliable results. In some embodiments, the patient population is two or more patients. For example, the patient population includes the population of patients described in Example 1. In other embodiments, the patient population is about 10 patients, about 50 patients, about 100 patients, about 150 patients, about 200 patients, about 250 patients, about 300 patients, about 350 patients, about 400 patients, about 450 patients, about 500 patients, about 550 patients, about 600 patients, about 650 patients, about 700 patients, about 750 patients, about 800 patients, about 850 patients, about 900 patients, about 950 patients, about 1000 patients, or more. In further embodiments, the patient population is about 50 or more patients. In still other embodiments, the patient population is about 100 or more patients. In yet further embodiments, the patient population is about 150 or more patients. In other embodiments, the patient population is about 200 or more patients. However, one skilled in the art would readily be able to determine how many patients would be needed to generate a statistically relevant result.

In some embodiments, the methods are effective in reducing the patient's PVR by about 10%, about 15%, about 20%, about 25%, about 30%, about 40%, about 50%, about 60%, about 70%, or more relative to a patient population at the same level of disease diagnosis that is not receiving treatment with macitentan or aprocitentan. In some embodiments, the methods are effective in reducing the patient's PVR by about 30% relative to a patient population at the same level of disease diagnosis that is not receiving treatment with macitentan or aprocitentan.

The methods further may be effective in reducing the patient's TPG by at least about 10% relative to a patient population that has the same disease and is at the same level of disease diagnosis that is not receiving treatment with macitentan or aprocitentan. In some embodiments, the methods are effective in reducing the patient's TPG by about 10%, about 15%, about 20%, about 25%, about 30%, about 40%, about 50%, about 60%, about 70%, or more relative to a patient population at the same level of disease diagnosis that is not receiving treatment with macitentan or aprocitentan.

The methods include administering a therapeutically effective amount of macitentan or aprocitentan. The term “therapeutically effective amount” as used herein means an amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In some embodiments, the therapeutically effective amount corresponds to the free base or free base morphological form of macitentan or aprocitentan. In other embodiments, the therapeutically effective amount corresponds to a salt, solvate, or hydrate of macitentan or aprocitentan. Unless otherwise noted, the amounts disclosed herein correspond to the free form of macitentan or aprocitentan, exclusive of, for example, solvent (such as in solvates or hydrates) or counterions (such as in pharmaceutically acceptable salts).

In certain embodiments, the therapeutically effective amount of macitentan is less than about 15 mg. In further embodiments, the therapeutically effective amount of macitentan is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In other embodiments, the therapeutically effective amount of macitentan is about 1 to about 15 mg, about 1 to about 10 mg, about 1 to about 5 mg, about 5 to about 15 mg, about 5 to about 10 mg, or about 10 to about 15 mg. In yet further embodiments, the therapeutically effective amount is about 5 to about 15 mg. In yet further embodiments, the therapeutically effective amount is about 10 mg.

Higher amounts of macitentan may also be used in the methods described herein. In some embodiments, the therapeutically effective amount of macitentan is at least about 15 mg. In further embodiments, the therapeutically effective amount of macitentan is about 15 mg, about 17.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 52.5 mg, about 60 mg, about 62.5 mg, about 65 mg, about 67.5 mg, about 70 mg, about 72.5 mg, about 75 mg, about 77.5 mg, about 80 mg, about 82.5 mg, about 85 mg, about 87.5 mg, about 90 mg, about 92.5 mg, about 95 mg, about 97.5 mg, about 100 mg, about 102.5 mg, about 105 mg, about 107.5, mg, about 110 mg, about 112.5 mg, about 115 mg, about 117.5 mg, about 120 mg, about 122.5 mg, about 125 mg, about 127.5 mg, about 130 mg, about 132.5 mg, about 135 mg, about 137.5 mg, about 140, about 142.5 mg, about 145 mg, about 147.5 mg, about 150, about 152.5 mg, about 155 mg, about 157.5 mg, about 160 mg, about 162.5 mg, about 165 mg, about 167.5 mg, about 170 mg, about 172.5 mg, about 175 mg, about 177.5 mg, about 180 mg, about 182.5 mg, about 185 mg, about 187.5 mg, about 190 mg, about 192.5 mg, about 195 mg, about 197.5 mg, about 200 mg, or greater. In other embodiments, the therapeutically effective amount of macitentan is about 25 to 40 mg, 25 to 45, 25 to 50 mg, 30 to 40 mg, 30 to 45 mg, 30 to 50 mg, 35 to 40 mg, 36 to 39 mg, 50 to about 90 mg, about 50 to about 85 mg, about 50 to about 80 mg, about 50 to about 75 mg, about 50 to about 70 mg, about 50 to about 65 mg, about 50 to about 60 mg, about 60 to about 90 mg, about 60 to about 85 mg, about 60 to about 80 mg, about 60 to about 75 mg, about 60 to about 70 mg, about 65 to about 90 mg, about 65 to about 85 mg, about 65 to about 80 mg, about 65 to about 75 mg, about 70 to about 90 mg, about 70 to about 85 mg, about 70 to about 80 mg, about 70 to about 75 mg, about 72 to about 78 mg, about 75 to about 90 mg, about 75 to about 85 mg, about 80 to about 90 mg, about 110 to about 200 mg, about 110 to about 150 mg, about 110 to about 160, about 125 to about 160 mg, about 125 to about 175 mg, about 145 to about 155 mg, about 140 to about 160 mg, or about 140 to about 175 mg. In yet further embodiments, the therapeutically effective amount of macitentan is about 60 to about 90 mg. In yet further embodiments, the therapeutically effective amount of macitentan is about 75 mg.

These amounts may be administered once or twice per day. In some aspects, these amounts are administered once per day. In other embodiments, the therapeutically effective amount of macitentan is about 25 to about 40 mg once per day. In further embodiments, the therapeutically effective amount of macitentan is about 25 to about 45 mg once per day. In still other embodiments, the therapeutically effective amount of macitentan is about 25 to about 50 mg once per day. In yet further embodiments, the therapeutically effective amount of macitentan is about 30 to about 40 mg once per day. In other embodiments, the therapeutically effective amount of macitentan is about 30 to about 45 mg once per day. In further embodiments, the therapeutically effective amount of macitentan is about 30 to about 50 mg once per day. In yet other embodiments, the therapeutically effective amount of macitentan is about 35 to about 40 mg once per day. In still further embodiments, the therapeutically effective amount of macitentan is about 36 to about 39 mg once per day. In other embodiments, the therapeutically effective amount of macitentan is about 37.5 mg once per day. In further embodiments, the therapeutically effective amount of macitentan is about 60 to about 80 mg once per day. In still other embodiments, the therapeutically effective amount of macitentan is about 60 to about 85 mg once per day. In yet further embodiments, the therapeutically effective amount of macitentan is about 60 to about 90 mg once per day. In other embodiments, the therapeutically effective amount of macitentan is about 65 to about 75 mg once per day. In further embodiments, the therapeutically effective amount of macitentan is about 65 to about 85 mg once per day. In yet other embodiments, the therapeutically effective amount of macitentan is about 65 to about 90 mg once per day. In still further embodiments, the therapeutically effective amount of macitentan is about 70 to about 80 mg once per day. In other embodiments, the therapeutically effective amount of macitentan is about 72 to about 78 mg once per day. In further embodiments, the therapeutically effective amount of macitentan is about 75 mg once per day. In still other embodiments, the therapeutically effective amount of macitentan is about 110 to about 200 mg once per day. In yet further embodiments, the therapeutically effective amount of macitentan is about 110 to about 150 mg once per day. In other embodiments, the therapeutically effective amount of macitentan is about 110 to about 160 mg once per day. In further embodiments, the therapeutically effective amount of macitentan is about 125 to about 160 mg once per day. In yet other embodiments, the therapeutically effective amount of macitentan is about 125 to about 175 mg once per day. In still further embodiments, the therapeutically effective amount of macitentan is about 145 to about 155 mg once per day. In other embodiments, the therapeutically effective amount of macitentan is about 140 to about 160 mg once per day. In further embodiments, the therapeutically effective amount of macitentan is about 140 to about 175 mg once per day. In yet other embodiments, the therapeutically effective amount of macitentan is about 150 mg per day once per day.

In further aspects, the therapeutically effective amounts are administered twice per day. In other embodiments, the therapeutically effective amount of macitentan is about 25 to about 40 mg twice per day. In further embodiments, the therapeutically effective amount of macitentan is about 25 to about 45 mg twice per day. In still other embodiments, the therapeutically effective amount of macitentan is about 25 to about 50 mg twice per day. In yet further embodiments, the therapeutically effective amount of macitentan is about 30 to about 40 mg twice per day. In other embodiments, the therapeutically effective amount of macitentan is about 30 to about 45 mg twice per day. In further embodiments, the therapeutically effective amount of macitentan is about 30 to about 50 mg twice per day. In yet other embodiments, the therapeutically effective amount of macitentan is about 35 to about 40 mg twice per day. In still further embodiments, the therapeutically effective amount of macitentan is about 36 to about 39 mg twice per day. In other embodiments, the therapeutically effective amount of macitentan is about 37.5 mg twice per day. In further embodiments, the therapeutically effective amount of macitentan is about 60 to about 80 mg twice per day. In still other embodiments, the therapeutically effective amount of macitentan is about 60 to about 85 mg twice per day. In yet further embodiments, the therapeutically effective amount of macitentan is about 60 to about 90 mg twice per day. In other embodiments, the therapeutically effective amount of macitentan is about 65 to about 75 mg twice per day. In further embodiments, the therapeutically effective amount of macitentan is about 65 to about 85 mg twice per day. In yet other embodiments, the therapeutically effective amount of macitentan is about 65 to about 90 mg twice per day. In still further embodiments, the therapeutically effective amount of macitentan is about 70 to about 80 mg twice per day. In other embodiments, the therapeutically effective amount of macitentan is about 72 to about 78 mg twice per day. In further embodiments, the therapeutically effective amount of macitentan is about 75 mg twice per day.

The methods also contemplate administering a therapeutically effective amount of aprocitentan. In certain embodiments, the therapeutically effective amount of aprocitentan is about 100 mg per day to about 1500 mg. In other embodiments, the therapeutically effective amount of aprocitentan is about 100 mg per day to about 1500 mg per day. In some embodiments, the therapeutically effective amount of aprocitentan is about 300 to 450 mg per day. In other embodiments, the therapeutically effective amount of aprocitentan is about 325 to about 425 mg per day. In further embodiments, the therapeutically effective amount of aprocitentan is about 350 to about 400 mg per day. In yet other embodiments, the therapeutically effective amount of aprocitentan is about 375 mg per day. In still further embodiments, the therapeutically effective amount of aprocitentan is about 300 to about 425 mg. In other embodiments, the therapeutically effective amount of aprocitentan is about 300 to about 400 mg. In further embodiments, the therapeutically effective amount of aprocitentan is about 300 to about 375 mg per day. In still other embodiments, the therapeutically effective amount of aprocitentan is about 325 to about 450 mg. In yet further embodiments, the therapeutically effective amount of aprocitentan is about 325 to about 375 mg per day. In other embodiments, the therapeutically effective amount of aprocitentan is about 360 to about 390 mg per day. In further embodiments, the therapeutically effective amount of aprocitentan is less than about 75 mg. In further embodiments, the therapeutically effective amount of aprocitentan is about 1 mg, about 10 mg, about 12.5 mg, about 15 mg, about 20 mg, about 25 mg, about 30 mg, about 35 mg, about 40 mg, about 45 mg, about 50 mg, about 55 mg, about 60 mg, about 65 mg, about 70 mg, or about 75 mg. In other embodiments, the therapeutically effective amount of aprocitentan is about 5 to about 75 mg, about 5 to about 50 mg, about 5 to about 25 mg, about 12.5 to about 75 mg, about 12.5 to about 50 mg, about 25 to about 75 mg, about 25 to about 50 mg, or about 50 to about 75 mg. In yet further embodiments, the therapeutically effective amount of aprocitentan is about 25 to about 75 mg. In yet further embodiments, the therapeutically effective amount of aprocitentan is about 50 mg.

The disclosure also provides for up-titrating the amounts of macitentan from smaller to larger amounts over a period of time. In some embodiments, a starting amount of macitentan is administered to the patient for a first period of time. The starting amount of macitentan is then up-titrated, i.e., increased, to subsequent amounts for subsequent periods of time. For example, a second amount of macitentan is administered for a second period of time. The second amount of macitentan is then up-titrated to a third amount of macitentan for a third period of time.

The starting amounts may be increased to subsequent amount(s) in increments as determined by the attending physician. In some embodiments, the increments are about 5 mg, about 10 mg, about 15 mg, about 20 mg, about 25 mg, about 30 mg, about 35 mg, about 40 mg, about 45 mg, or about 50 mg. The starting and subsequent amounts of macitentan may be any therapeutically effective amount referenced in the disclosure. In some embodiments, the starting amount of macitentan is about 10 mg per day. In other embodiments, the subsequent amounts may be about 25 mg to about 50 mg per day or about 60 mg to about 90 mg of macitentan. In further embodiments, the first subsequent amount of macitentan that follows the starting amount of macitentan is about 25 mg to about 50 mg per day. In other embodiments, the second subsequent amount of macitentan that follows the first subsequent amount of macitentan is about 60 mg to about 90 mg. For example, the starting amount of macitentan is about 10 mg per day, which is up-titrated to about 25 mg to about 50 mg per day of macitentan, which is up-titrated to about 60 mg to about 90 mg per day of macitentan.

In some embodiments, the starting amount of macitentan is about 10 mg for a first period of time. For example, the first period of time is about 1 or 2 days, preferably 2 days. The starting amount of macitentan is then up-titrated to a second amount of macitentan of about 37.5 mg of macitentan for a second period of time. For example, the second period of time is about 2 to 4 days, preferably 3 days. The second amount of macitentan is then up-titrated to a third amount of macitentan of about 75 mg of macitentan for a third period of time. For example, the third period of time is about 6 to about 10 days, preferably 8 days. In other embodiments, 10 mg is administered to the patient for 2 consecutive days, followed by about 37.5 mg of macitentan for 3 consecutive days, and followed by about 75 mg of macitentan for 8 days.

In some embodiments, about 10 mg per day of macitentan is administered for about 15 to about 45 days. In other embodiments, about 25 mg to about 50 mg per day of macitentan is administered for about 15 to about 45 days. In further embodiments, the amount of macitentan is up-titrated from about 10 mg per day, followed by about 25 mg to about 50 mg per day, and followed by about 60 mg to about 90 mg per day.

The following aspects (a)-(l) and (a′)-(l′) describe aspects for dosing macitentan or aprocitentan. In some embodiments, aspects (a)-(l) and (a′)-(l′) relate to methods for treating pulmonary hypertension in a patient with left ventricular assist device (LVAD) implantation by administering macitentan. In other embodiments, aspects (a)-(l) and (a′)-(l′) relate to methods for treating pulmonary hypertension in a patient with left ventricular assist device (LVAD) implantation by administering aprocitentan. In further embodiments, aspects (a)-(l) and (a′)-(l′) relate to methods for improving cardiac transplant eligibility in a patient with left ventricular assist device (LVAD) implantation using macitentan. In still other embodiments, aspects (a)-(l) and (a′)-(l′) relate to methods for improving cardiac transplant eligibility in a patient with left ventricular assist device (LVAD) implantation using aprocitentan.
(a) In some aspects, the amount of macitentan is from 20 mg per day to 300 mg per day. The amount may also be more than 20 mg per day to 300 mg per day. The amount may be 25 mg per day to 300 mg per day. For example, the amount is equal to or more than 20 mg per day to 250 mg per day. Preferably, the amount is 25 mg to 200 mg per day. According to a more preferred aspect, these amounts are applied once a day.
(b) In other aspects, such as according to aspect (a), the amount of macitentan is 60 to 90 mg per day. Preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day. Further, the dosage of macitentan may be escalated from 10 mg per day, followed by 25 to 50 mg per day, preferably 37.5 mg per day, followed by 60 to 90 mg per day, preferably 75 mg per day, optionally followed by 100 to 200 mg per day, preferably 150 mg per day. According to a more preferred aspect, these amounts are applied once a day.
(c) In further aspects, such as according to aspect (a), the amount of macitentan is 25 to 50 mg per day. Preferably, the amount is 30 to 45 mg per day, more preferably 35 to 40 mg per day and most preferably 37.5 mg per day. It is to be understood that each of the lower limits disclosed above may be combined with each of the upper limits, i.e. the amount could also be from 25 to 45 mg per day, or from 25 to 40 mg per day. Also disclosed are amounts from 30 to 50 mg, or 30 to 40 mg per day. Further preferred ranges are 36 to 39 mg per day. According to a more preferred aspect, these amounts are applied once a day.
(d) In other aspects, such as according to aspect (a), the amount of macitentan is 110 to 200 mg per day. Preferably, the amount is 125 to 175 mg per day, more preferably 140 to 160 mg per day and most preferably 150 mg per day. It is to be understood that each of the lower limits disclosed above may be combined with each of the upper limits, i.e. the amount could also be from 110 to 175 mg per day, from 110 to 160 mg per day or from 110 to 150 mg per day. Also disclosed are amounts from 125 to 160 mg or 140 to 175 mg per day. Further preferred ranges are 145 to 155 mg per day. According to a more preferred aspect, these amounts are applied once a day.
(e) In further aspects, such as according to aspect (a), the amount of macitentan is 60 to 90 mg twice per day. Preferably, the amount is 65 to 85 mg twice per day, more preferably 70 to 80 mg twice per day and most preferably 75 mg twice per day. It is to be understood that each of the lower limits disclosed above may be combined with each of the upper limits, i.e. the amount could also be from 60 to 85 mg twice per day, from 60 to 80 mg twice per day, or from 60 to 75 mg twice per day. Also disclosed are amounts from 65 to 90 mg twice per day, or 65 to 75 mg twice per day. Further preferred ranges are 72 to 78 mg twice per day.
(f) In other aspects, such as according to aspect (a), the amount of macitentan is 25 to 50 mg twice per day, preferably 30 to 45 mg twice per day, more preferably 35 to 40 mg twice per day and most preferably 37.5 mg twice per day. It is to be understood that each of the lower limits disclosed above may be combined with each of the upper limits, i.e. the amount could also be from 25 to 45 mg twice per day, or from 25 to 40 mg twice per day. Also disclosed are amounts from 30 to 50 mg twice per day, or 30 to 40 mg twice per day. Further preferred ranges are 36 to 39 mg twice per day.
(g) In further aspects, such as according to aspect (a), the amount of macitentan is escalated from 10 mg per day, followed by 25 to 50 mg per day, preferably 37.5 mg per day, and optionally followed by 60 to 90 mg per day, preferably 75 mg per day. Therein “followed by 25 to 50 mg per day” means preferably, the amount is 30 to 45 mg per day, more preferably 35 to 40 mg per day and most preferably 37.5 mg per day. It is to be understood that each of the lower limits disclosed may be combined with each of the upper limits, i.e. the amount could also be from 25 to 45 mg per day, or from 25 to 40 mg per day. Also disclosed are amounts from 30 to 50 mg, or 30 to 40 mg per day. Further preferred ranges are 36 to 39 mg per day. It is to be understood that any escalation to a higher amounts may be followed by return to the lower amount in case the higher amount is not tolerated. The phrase “optionally followed by 60 to 90 mg per day” means preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day. It is to be understood that any escalation to a higher amount may be followed by return to the lower amount in case the higher amount is not tolerated. According to a more preferred aspect, these amounts are applied once a day.

In yet further aspects, the amount of macitentan is 10 mg per day. Patients obtaining this amount may receive an immediate amount escalation to 37.5 mg per day, optionally followed by 75 mg per day.

In yet other aspects, such as according to aspect (a), the amount of macitentan is escalated from 10 mg per day, followed by 25 to 50 mg per day, preferably 37.5 mg per day, optionally followed by 60 to 90 mg per day, preferably 75 mg per day, optionally followed by 110 to 200 mg per day, preferably 150 mg per day. The phrase “optionally followed by 110 to 200 mg per day” means preferably, the amount is 125 to 175 mg per day, more preferably 140 to 160 mg per day and most preferably 150 mg per day. It is to be understood that each of the lower limits disclosed above may be combined with each of the upper limits, i.e. the amount could also be from 110 to 175 mg per day, from 110 to 160 mg per day or from 110 to 150 mg per day. Also disclosed are amounts from 125 to 160 mg or 140 to 175 mg per day. Further preferred ranges are 145 to 155 mg per day. According to a more preferred aspect, these amounts are applied once a day.
(h) In further aspects, such as according to aspect (a), the amount of macitentan is escalated from 10 mg per day, followed by 60 to 90 mg per day, preferably 75 mg once per day or 37.5 mg twice a day. The phrase “optionally followed by 60 to 90 mg per day” means preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day. It is to be understood that any escalation to a higher amount may be followed by return to the lower amount in case the higher amount is not tolerated. According to a more preferred aspect, these amounts are applied once a day.

In other aspects, such as according to aspect (a), the amount of macitentan is escalated from 10 mg per day, followed by 60 to 90 mg per day, preferably 75 mg once per day or 37.5 mg twice a day, optionally followed by 110 to 200 mg per day, preferably 150 mg per day. The phrase “optionally followed by 110 to 200 mg per day” means preferably, the amount is 125 to 175 mg per day, more preferably 140 to 160 mg per day and most preferably 150 mg per day. It is to be understood that each of the lower limits disclosed above may be combined with each of the upper limits, i.e. the amount could also be from 110 to 175 mg per day, from 110 to 160 mg per day or from 110 to 150 mg per day. Also disclosed are amounts from 125 to 160 mg or 140 to 175 mg per day. Further preferred ranges are 145 to 155 mg per day. According to a more preferred aspect, these amounts are applied once a day.

In yet other aspects, the amount of macitentan is 10 mg per day. Patients obtaining this amount may receive an immediate amount escalation to 75 mg per day or to 150 mg per day.
(i) In further aspects, such as according to aspect (g), the amount of macitentan is escalated from 10 mg once per day, preferably for 15 to 45 days; followed by 25 to 50 mg per day, preferably 37.5 mg once per day, preferably for 15 to 45 days; and optionally followed by 60 to 90 mg per day, preferably by 75 mg once per day or 37.5 mg twice per day. It is to be understood that any escalation to a higher amount may be followed by return to the lower amount in case the higher amount is not tolerated. Therein, the term “15 to 45 days” means preferably 20 to 40 days, more preferably 21 to 35 days, and most preferably 28 to 30 days, i.e. about one month. Moreover, the phrase “followed by 25 to 50 mg per day” means preferably, the amount is 30 to 45 mg per day, more preferably 35 to 40 mg per day and most preferably 37.5 mg per day. It is to be understood that each of the lower limits disclosed may be combined with each of the upper limits, i.e. the amount could also be from 25 to 45 mg per day, or from 25 to 40 mg per day. Also disclosed are amounts from 30 to 50 mg, or 30 to 40 mg per day. Further preferred ranges are 36 to 39 mg per day. Moreover, the phrase “followed by 60 to 90 mg per day” means preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day, or 36 to 39 mg twice per day. According to a more preferred aspect, these amounts are applied once a day.

In other aspects, such as according to aspect (g), the amount of macitentan is escalated from 10 mg once per day, preferably for 15 to 45 days; followed by 25 to 50 mg per day, preferably 37.5 mg once per day, preferably for 15 to 45 days; optionally followed by 60 to 90 mg per day, preferably by 75 mg once per day or 37.5 mg twice per day, preferably for 15 to 45 days; optionally followed by 110 to 200 mg per day, preferably 150 mg per day. The phrase “optionally followed by 110 to 200 mg per day” means preferably, the amount is 125 to 175 mg per day, more preferably 140 to 160 mg per day and most preferably 150 mg per day. It is to be understood that each of the lower limits disclosed above may be combined with each of the upper limits, i.e. the amount could also be from 110 to 175 mg per day, from 110 to 160 mg per day or from 110 to 150 mg per day. Also disclosed are amounts from 125 to 160 mg or 140 to 175 mg per day. Further preferred ranges are 145 to 155 mg per day. According to a more preferred aspect, these amounts are applied once a day.
(j) In further aspects, such as according to aspect (h), the amount of macitentan is escalated from 10 mg once per day, preferably for 15 to 45 days; followed by 60 to 90 mg per day, preferably by 75 mg once per day or 37.5 mg twice per day. Therein, the term “15 to 45 days” means preferably 20 to 40 days, more preferably 21 to 35 days, and most preferably 28 to 30 days, i.e. about one month. The phrase “followed by 60 to 90 mg per day” means preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day, or 36 to 39 mg twice per day.

In other aspects, such as according to aspect (h), the amount of macitentan is escalated from 10 mg once per day, preferably for 15 to 45 days; followed by 60 to 90 mg per day, preferably by 75 mg once per day or 37.5 mg twice per day, preferably for 15 to 45 days; optionally followed by 110 to 200 mg per day, preferably 150 mg per day. The phrase “optionally followed by 110 to 200 mg per day” means preferably, the amount is 125 to 175 mg per day, more preferably 140 to 160 mg per day and most preferably 150 mg per day. It is to be understood that each of the lower limits disclosed above may be combined with each of the upper limits, i.e. the amount could also be from 110 to 175 mg per day, from 110 to 160 mg per day or from 110 to 150 mg per day. Also disclosed are amounts from 125 to 160 mg or 140 to 175 mg per day. Further preferred ranges are 145 to 155 mg per day. According to a more preferred aspect, these amounts are applied once a day.
(k) In further aspects, such as according to any one of aspects (a) to (c), the amount of macitentan is escalated to 25 to 50 mg per day, preferably 37.5 mg per day; preferably for 15 to 45 days; optionally followed by 60 to 90 mg per day, preferably by 75 mg per day. It is to be understood that any escalation to a higher amount may be followed by return to the lower amount in case the higher amount is not tolerated. Therein, the term “15 to 45 days” means preferably 20 to 40 days, more preferably 21 to 35 days, and most preferably 28 to 30 days, i.e. about one month. Moreover, the phrase “followed by 25 to 50 mg per day” means preferably, the amount is 30 to 45 mg per day, more preferably 35 to 40 mg per day and most preferably 37.5 mg per day. It is to be understood that each of the lower limits disclosed may be combined with each of the upper limits, i.e. the amount could also be from 25 to 45 mg per day, or from 25 to 40 mg per day. Also disclosed are amounts from 30 to 50 mg, or 30 to 40 mg per day. Further preferred ranges are 36 to 39 mg per day. Moreover, the phrase “followed by 60 to 90 mg per day” means preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day, or 36 to 39 mg twice per day. It is to be understood that, optionally, the amount of macitentan can be further raised to reach from 110 to 200 mg per day. The amount is thereby escalated as in aspect (h), (i) or (j). According to a more preferred aspect, these amounts are applied once a day.
(l) In other aspects, such as according to aspect (a) or (b), the amount of macitentan is 60 to 90 mg per day, preferably 75 mg per day. The phrase “60 to 90 mg per day” means preferably, the amount is 65 to 85 mg per day, more preferably the amount is 70 to 80 mg per day and most preferably the amount is 75 mg per day. It is to be understood that each of the lower limits disclosed above may be combined with each of the upper limits, i.e. the amount could also be from 60 to 85 mg, from 60 to 80 mg, or from 60 to 75 mg per day. Also disclosed are amounts from 65 to 90 mg, or 65 to 75 mg per day. Further preferred ranges are 72 to 78 mg per day, or 36 to 39 mg twice per day. It is to be understood, that optionally, the amount of macitentan can be further raised to reach from 110 to 200 mg per day. The amount is thereby escalated as in aspect (h), (i) or (j). According to a more preferred aspect, these amounts are applied once a day.
(a′) In certain aspects, the amount of macitentan is from 20 mg per day to 300 mg per day. It is to be understood that the disclosure of aspect (a) applies analogously.
(b′) In other aspects, such as according to aspect (a′), the amount of macitentan is 60 to 90 mg per day, preferably 65 to 85 mg per day, more preferably 70 to 80 mg per day and most preferably 75 mg per day. It is to be understood that the disclosure of aspect (b) applies analogously.
(c′) In further aspects, such as according to aspect (a′), the amount of macitentan is 25 to 50 mg per day, preferably 30 to 45 mg per day, more preferably 35 to 40 mg per day and most preferably 37.5 mg per day. It is to be understood that the disclosure of aspect (c) applies analogously.
(d′) In other aspects, such as according to aspect (a′), the amount of macitentan is 110 to 200 mg per day, preferably 125 to 175 mg per day, more preferably 140 to 160 mg per day and most preferably 150 mg per day. It is to be understood that the disclosure of aspect (d) applies analogously.
(e′) In further aspect, such as according to aspect (a′), the amount of macitentan is 60 to 90 mg twice per day, preferably 65 to 85 mg twice per day, more preferably 70 to 80 mg twice per day and most preferably 75 mg twice per day. It is to be understood that the disclosure of aspect (e) applies analogously.
(f′) In other aspect, such as according to aspect (a′), the amount of macitentan is 25 to 50 mg twice per day, preferably 30 to 45 mg twice per day, more preferably 35 to 40 mg twice per day and most preferably 37.5 mg twice per day. It is to be understood that the disclosure of aspect (f) applies analogously.
(g′) In other aspects, such as according to aspect (a′), the amount of macitentan is escalated from 10 mg per day, followed by 25 to 50 mg per day, preferably 37.5 mg per day, and optionally followed by 60 to 90 mg per day, preferably 75 mg per day. It is to be understood that the disclosure of aspect (g) applies analogously.
(h′) In further aspects, such as according to aspect (a′), the amount of macitentan is escalated from 10 mg per day, followed by 60 to 90 mg per day, preferably 75 mg once per day or 37.5 mg twice a day. It is to be understood that the disclosure of aspect (h) applies analogously.
(i′) In other aspects, such as according to aspect (g′), the amount of macitentan is escalated from 10 mg once per day, preferably for 15 to 45 days; followed by 25 to 50 mg per day, preferably 37.5 mg once per day, preferably for 15 to 45 days; and optionally followed by 60 to 90 mg per day, preferably by 75 mg once per day or 37.5 mg twice per day. It is to be understood that the disclosure of aspect (i) applies analogously.
(j′) In further aspects, such as according to aspect (h′), the amount of macitentan is escalated from 10 mg once per day, preferably for 15 to 45 days; followed by 60 to 90 mg per day, preferably by 75 mg once per day or 37.5 mg twice per day. It is to be understood that the disclosure of aspect (j) applies analogously.
(k′) In other aspects, such as according to any one of aspects (a′) to (c′), the amount of macitentan is escalated from 25 to 50 mg per day, preferably 37.5 mg per day, preferably for 15 to 45 days; optionally followed by 60 to 90 mg per day, preferably by 75 mg per day. It is to be understood that the disclosure of aspect (k) applies analogously.
(l′) In further aspects, such as according to aspect (a′) or (b′), the amount of macitentan is 60 to 90 mg per day, preferably 75 mg per day. It is to be understood that the disclosure of aspect (1) applies analogously.

It is to be understood that the comments and details of aspects (a) to (1) also apply to aspects (a′) to (l′).

According to a further aspect of the present invention, in each of the above-mentioned aspects, that is, in each of aspects (a) to (1) and (a′) to (l′), macitentan can be replaced by aprocitentan, wherein the weight amounts of macitentan will be replaced by a 5-fold weight amount of aprocitentan, wherein the amount of aprocitentan is 300 to 450 mg per day. Preferably, the amount of aprocitentan is 325 to 425 mg per day, more preferably the amount of aprocitentan is 350 to 400 mg per day and most preferably the amount of aprocitentan is 375 mg per day. It is to be understood that each of the lower limits disclosed above may be combined with each of the upper limits, i.e. the amount of aprocitentan could also be from 300 to 425 mg, from 300 to 400 mg, or from 300 to 375 mg per day. Also disclosed are amounts of aprocitentan from 325 to 450 mg, or 325 to 375 mg per day. Further preferred ranges are 360 to 390 mg of aprocitentan per day. According to a more preferred aspect, these amounts of aprocitentan are applied once a day.

The methods described herein may also include discontinuing administration of another pharmaceutical agent that is useful in the treatment of pulmonary hypertension. In some embodiments, the methods include discontinuing treatment with an endothelin receptor antagonist (ERA) that is not macitentan or aprocitentan prior to administration of macitentan or aprocitentan. For example, the ERA is bosentan or ambrisentan, or a pharmaceutically acceptable salt thereof. In some aspects, the ERA is bosentan. In other aspects, the ERA is ambrisentan.

In other embodiments, the methods include discontinuing treatment with a phosphodiesterase type 5 (PDE-5) inhibitor prior to administration of macitentan or aprocitentan. For example, the PDE-5 inhibitor is tadalafil, sildenafil, vardenafil, or udenafil, or a pharmaceutically acceptable salt thereof. In some embodiments, the PDE-5 inhibitor is sildenafil. In further embodiments, the PDE-5 inhibitor is vardenafil. In other embodiments, the PDE-5 inhibitor is udenafil. In still further embodiments, the PDE-5 inhibitor is tadalafil. Tadalafil is commercially available as Adcirca™ and has the following structure.

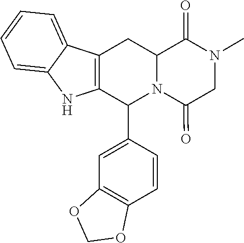 [SMILES: CN1CC(=O)N2C(Cc3c([nH]c4ccccc34)C2c2ccc3c(c2)OCO3)C1=O]

In further embodiments, the methods include discontinuing treatment with other PAH-specific therapies prior to administration of macitentan or aprocitentan. For example, other PAH-specific therapies include inhaled nitric oxide (iNO), soluble guanylate cyclase (sGC) stimulators, or oral prostanoids (e.g., intravenous or subcutaneous), among others. In some embodiments, the PAH-specific therapy is iNO. In other aspects, the PAH specific therapy is a sGC stimulator. One example of a sGC stimulator is riociguat. Riociguat is commercially available as Adempas™ and has the following structure:

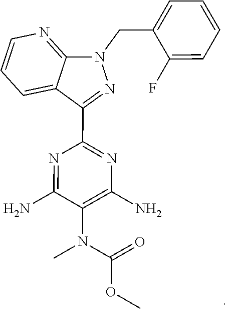 [SMILES: COC(=O)N(C)c1c(N)nc(-c2nn(Cc3ccccc3F)c3ncccc23)nc1N]

In patients who were on other PAH therapies, the methods may also include a washout period prior to administering macitentan or aprocitentan. The term “washout period” as used herein refers to a period of time whereby no other PAH therapy is continued. In some embodiments, the washout period is at least one day, or preferably at least one week.

The methods described herein are effective in the lowering of PVR. In some embodiments, the PVR is reduced by at least about 10% relative to a patient population at the same level of disease diagnosis (placebo group) that is not receiving treatment with macitentan or aprocitentan. In other embodiments, the methods result in the lowering of PVR levels to less than about 4 WU. In further embodiments, the methods result in lowering the PVR to less than about 3 WU. In yet other embodiments, the methods result in lowering the PVR to less than about 2 WU, or about 1 WU.

As used herein, unless otherwise noted, macitentan is defined as propylsulfamic acid [5-(4-bromo-phenyl)-6-[2-(5-bromo-pyrimidin-2-yloxy)-ethoxy]-pyrimidin-4-yl]-amide:

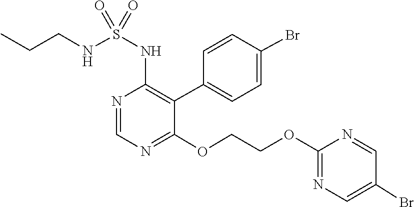 [SMILES: CCCNS(=O)(=O)Nc1ncnc(OCCOc2ncc(Br)cn2)c1-c1ccc(Br)cc1]

or a pharmaceutically acceptable salt, solvate, hydrate or morphological form thereof. In some embodiments, the disclosure is directed to the macitentan free base. In other embodiments, the disclosure is directed to macitentan salts. In further embodiments, the disclosure is directed to macitentan solvates. In yet other embodiments, the disclosure is directed to macitentan hydrates. In yet further embodiments, the disclosure is directed to macitentan morphological forms.

Macitentan is an endothelin receptor antagonist (ERA) that acts as an antagonist of two endothelin (ET) receptor subtypes, ETA and ETB (Kholdani et al, Macitentan for the treatment of pulmonary arterial hypertension. Vasc. Health Risk Manag. (2014), 10, 665-673). Currently, macitentan is taken as a 10 mg oral dose once a day. Its half-life in humans is about 16 hours and steady state is reached by the third day of administration (Bruderer et al., Absorption, distribution, metabolism, and excretion of macitentan, a dual endothelin receptor antagonist, in humans. Xenobiotica (2012), 42(9), 901-910). It is absorbed slowly into the plasma (Sidharta et al., Macitentan: entry-into-humans study with a new endothelin receptor antagonist. Eur. J. Clin. Pharmacol. (2011), 67, 977-984). Macitentan dealkylates into the active metabolite ACT-132577, i.e. aprocitentan, which reaches its peak plasma concentration about 30 hours after the first dose is administered, and it has a half-life of approximately 48 hours. Although ACT-132577 has a lower affinity for the ET receptors than its parent compound (Iglarz et al., Pharmacology of macitentan, an orally active tissue-targeting dual endothelin receptor antagonist. J. Pharmacol. Exp. Ther. (2008), 327(3), 736-745), it maintains higher plasma concentrations than macitentan. Both compounds can be excreted from the body through the urine or feces.

According to further aspects of the disclosure, macitentan can be replaced by its active metabolite, known under the code name ACT-132577 and the international non-proprietary name aprocitentan:

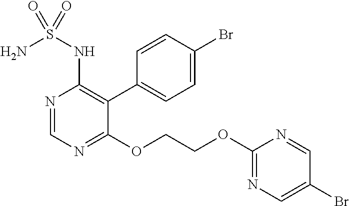 [SMILES: NS(=O)(=O)Nc1ncnc(OCCOc2ncc(Br)cn2)c1-c1ccc(Br)cc1]

whereby any weight amount of macitentan will be replaced by a 5-fold weight amount of aprocitentan. In some embodiments, the disclosure is directed to the aprocitentan free base. In other embodiments, the disclosure is directed to aprocitentan salts. In further embodiments, the disclosure is directed to aprocitentan solvates. In yet other embodiments, the disclosure is directed to aprocitentan hydrates. In yet further embodiments, the disclosure is directed to aprocitentan morphological forms.

As used herein, “morphological forms” refers to amorphous or crystalline forms of macitentan or aprocitentan. In some embodiments, the macitentan or aprocitentan is a crystalline form. In other embodiments, macitentan or aprocitentan is an amorphous form. The crystallinity may be determined by those skilled in the art using one or more techniques such as, e.g., single crystal x-ray diffraction, powder x-ray diffraction, differential scanning calorimetry, melting point, among others.

As used herein, “hydrate” includes forms of macitentan or aprocitentan whereby one or more molecules of water are bound through intermolecular forces or chemical bonds to one or more locations of the macitentan or aprocitentan molecule.

“Solvate” as used herein refers to forms of macitentan or aprocitentan whereby one or more solvent molecules are bound through intermolecular forces or chemical bonds to one or more locations of the macitentan or aprocitentan molecule.

Pharmaceutically acceptable salts of macitentan or aprocitentan may readily be selected by those skilled in the art. The expression pharmaceutically acceptable salts encompasses either salts with inorganic acids or organic acids like hydrohalogenic acids, e.g. hydrochloric or hydrobromic acid; sulfuric acid, phosphoric acid, nitric acid, citric acid, formic acid, acetic acid, maleic acid, tartaric acid, methylsulfonic acid, p-toluolsulfonic acid and the like or in case macitentan or aprocitentan is acidic in nature with an inorganic base like an alkali or earth alkali base, e.g. sodium hydroxide, potassium hydroxide, calcium hydroxide and the like.

As used herein, unless otherwise noted, the terms “treating”, “treatment” and the like, shall include the management and care of a patient for the purpose of combating a disease, condition, or disorder. The terms “treating” and “treatment” also include the administration of macitentan or aprocitentan or pharmaceutical compositions as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder.

The terms “subject” and “patient” are interchangeably used herein to refer to an animal, preferably a mammal, most preferably a human, who has been the object of treatment.

The methods also permit administering a concomitant standard of care or background therapy. The term “standard of care” typically refers to a physician prescribed treatment of the disease condition at issue. In some embodiments, the standard of care comprises, consists of, or consists essentially of administering an additional pharmaceutical agent that is effective for treating PH. The standard of care may be administered to the patient prior to, subsequently to, or concurrently with macitentan or aprocitentan. In some embodiments, the standard of care is administered before macitentan or aprocitentan. In other embodiments, the standard of care is administered after macitentan or aprocitentan. In further embodiments, the standard of care is administered concurrently with macitentan or aprocitentan. In yet other embodiments, the standard of care is present for at least three months at a stable dose prior to administration of macitentan or aprocitentan.

In some embodiments, the standard of care is a PDE5 inhibitor. Examples of PDE5 inhibitors include, without limitation, sildenafil, tadalafil, vardenafil, and udenafil, preferably tadalafil. In certain embodiments that include a PDE5 inhibitor standard of care, sildenafil is not used. In other embodiments, the standard of care is a prostacyclin analogue. Examples of prostacyclin analogues include, without limitation, epoprostenol, treprostinil, iloprost, and beraprost. In further embodiments, the standard of care is a prostacyclin receptor agonist. Examples of useful prostacyclin receptor agonists include, without limitation, selexipag and ralinepag, preferably selexipag. In yet other embodiments, the standard of care is a soluble guanylate cyclase stimulator. Examples of soluble guanylate cyclase stimulators include, without limitation, riociguat and vericiguat.

In the methods described herein, the therapeutically effective amount of macitentan or aprocitentan is safe, effective, or safe and effective. As used herein, unless otherwise noted, the term “safe” shall mean without undue adverse side effects (such as toxicity, irritation, or allergic response), commensurate with a reasonable benefit/risk ratio when used in the manner of this invention. Similarly, unless otherwise noted, the term “effective” means the efficacy of treatment has been demonstrated for the treatment of patients with PH when dosed in a therapeutically effective dose. In certain embodiments, the methods described herein are safe. In other embodiments, the methods described herein are effective. In further embodiments, the methods described herein are safe and effective. In yet other embodiments, the therapeutically effective amount of macitentan or aprocitentan is safe. In still further embodiments, the therapeutically effective amount of macitentan or aprocitentan is effective. In other embodiments, the therapeutically effective amount of macitentan or aprocitentan is safe and effective.

### Formulations/Compositions

Pharmaceutical compositions containing macitentan or aprocitentan as the active ingredient can be prepared by intimately mixing macitentan or aprocitentan with a pharmaceutical carrier according to conventional pharmaceutical compounding techniques. As used herein, the terms “composition” and “formulation” are used interchangeably and encompass a product comprising the specified ingredients in the specified amounts, as well as any product, such as a pharmaceutical product, which results, directly or indirectly, from combinations of the specified ingredients in the specified amounts. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), herein incorporated by reference for such disclosure.

The pharmaceutical compositions or pharmaceutical drug products may be administered by a number of routes as determined by those skilled in the art. Preferably, the pharmaceutical compositions or drug products are administered by route that is suitable for macitentan or aprocitentan. In some embodiments, the pharmaceutical compositions or drug products are administered orally, rectally, parenterally, e.g., by intravenous, intramuscular, subcutaneous, intrathecal or transdermal administration or sublingually or as ophthalmic preparation or administered as aerosol, preferably orally. Preferably, the pharmaceutical compositions or drug products are administered orally. Examples of applications are capsules, tablets, orally administered suspensions or solutions, suppositories, injections, eye-drops, ointments or aerosols/nebulizers.

The preferred application is oral administration. The dosage used depends upon the type of the specific active ingredient, the age and the requirements of the patient and the kind of application. Generally, dosages of 0.001-0.25 mg/kg body weight per day are considered for an average body weight of about 70 kg. The preparations can contain inert or as well pharmacodynamically active excipients. Tablets or granules, for example, could contain a number of binding agents, filling excipients, carrier substances or diluents.

The compositions may be administered in enteral or oral form e.g. as tablets, dragees, gelatin capsules, emulsions, solutions or suspensions, in nasal form like sprays or rectally in form of suppositories. The compositions may also be administered intramuscularly, parenterally or intravenously, e.g., in form of injectable solutions.

The pharmaceutical compositions may contain macitentan or aprocitentan in combination with inorganic and/or organic excipients which are usual in the pharmaceutical industry such as lactose, maize or derivatives thereof, talcum, stearinic acid or salts of these materials.

For gelatin capsules vegetable oils, waxes, fats, liquid or half-liquid polyols may be used. For the preparation of solutions and syrups e.g. water, polyols, saccharose, glucose can be used. Injectables can be prepared by using e.g. water, polyols, alcohols, glycerin, vegetable oils, lecithin or liposomes. Suppositories may be prepared by using natural or hydrogenated oils, waxes, fatty acids (fats), liquid or half-liquid polyols.

The compositions may contain in addition preservatives, stability improving substances, viscosity improving or regulating substances, solubility improving substances, sweeteners, dyes, taste improving compounds, salts to change the osmotic pressure, buffer or anti-oxidants.

To prepare such pharmaceutical compositions, macitentan or aprocitentan, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective amount as described above. The pharmaceutical compositions herein will contain, per unit dosage unit, e.g., tablet, capsule, powder, injection, suppository, teaspoonful and the like, the therapeutically effective amounts of macitentan or aprocitentan as disclosed herein. The dosages, however, may be varied depending upon the requirement of the patients, the severity of the condition being treated and the macitentan or aprocitentan employed. The use of either daily administration or post-periodic dosing may be employed.

Preferably, the pharmaceutical compositions are in unit dosage forms such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. For preparing solid compositions such as tablets, the principal active ingredient (e.g., macitentan or aprocitentan) is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of macitentan or aprocitentan. In certain embodiments, two active ingredients can be formulated together, e.g., in a bi-layer tablet formulation. When referring to these preformulation compositions as homogeneous, it is meant that the active ingredients are dispersed evenly throughout the composition so that the composition may be readily subdivided into equally effective dosage forms such as tablets, pills and capsules. This solid preformulation composition is then subdivided into unit dosage forms of the type described above containing from any one of the therapeutically effective amounts of macitentan or aprocitentan. The tablets or pills of the composition can be coated or otherwise compounded to provide a dosage form affording the advantage of prolonged action. For example, the tablet or pill can comprise an inner dosage and an outer dosage component, the latter being in the form of an envelope over the former.

The liquid forms in which the compositions of the present disclosure may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described herein may also be carried out using a pharmaceutical composition comprising macitentan or aprocitentan and a pharmaceutically acceptable carrier. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, macitentan or aprocitentan may be administered in a single daily dose, or the total daily dosage may be administered in divided doses of two, three or four times daily. In some embodiments, the macitentan or aprocitentan is administered orally in the form of a tablet once daily. Preferably, macitentan or aprocitentan is administered in a single dose, or more preferably a once-daily tablet.

For instance, for oral administration in the form of a tablet or capsule, the active drug component (e.g., macitentan or aprocitentan) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare pharmaceutical compositions of the present disclosure, macitentan or aprocitentan, as the active ingredient, may be intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

The following Examples are provided to illustrate some of the concepts described within this disclosure. While the Examples are considered to provide an embodiment, it should not be considered to limit the more general embodiments described herein. Further, in the following example, efforts have been made to ensure accuracy with respect to numbers used (e.g., amounts, temperature, etc.) but some experimental error and deviation should be accounted for.

ABBREVIATIONS

| | |
|---|---|
| 2D | Two-dimensional |
| 3D | Three-dimensional |
| 6MWD | 6-minute walk distance |
| A′ | Tricuspid peak diastolic annular velocity a′ |
| AE | Adverse event |
| ALT | Alanine aminotransferase |
| ANCOVA | Analysis of covariance |
| AST | Aspartate aminotransferase |
| BMI | Body mass index |
| BP | Blood pressure |
| BSA | Body surface area |
| BUN | Blood urea nitrogen |
| CI | Cardiac index |
| CL | Confidence limit |
| CO | Cardiac output |
| CVs | Coefficients of variation |
| CYP3A4 | Cytochrome P450 isozyme 3A4 |
| DBP | Diastolic blood pressure |
| E′ | Tricuspid peak diastolic annular velocity e′ |
| ECG | Electrocardiogram |
| EOS | End-of-Study |
| EOT | End-of-Treatment |
| ERA | Endothelin receptor antagonist |
| ET-1 | Endothelin-1 |
| ET_{A} | Endothelin receptor A |
| ET_{B} | Endothelin receptor B |
| FAS | Full Analysis Set |
| FC | Functional class |
| FEV₁ | Forced expiratory volume in 1 second |
| FVC | Forced vital capacity |
| GDF-15 | Growth differentiation factor-15 |
| GFR | Glomerular filtration rate |
| GMR | Geometric mean ratio |
| HF | Heart failure |
| hsCRP | High sensitivity C-reactive protein |
| hs-cTnT | High sensitivity cardiac troponin T |
| i.v. | Intravenous |
| ICH | International Conference On Harmonisation |
| IL-6 | Interleukin-6 |
| iNO | Inhaled nitric oxide |
| LDH | Lactate dehydrogenase |
| LVAD | Left ventricular assist device |
| MedDRA | Medical Dictionary For Regulatory Activities |
| mPAP | Mean pulmonary arterial pressure |
| mRAP | Mean right atrial pressure |
| NGAL | Neutrophil gelantinase-associated lipocalin |
| NT-proBNP | N-terminal prohormone of brain natriuretic peptide |
| PAH | Pulmonary arterial hypertension |
| PAWP | Pulmonary artery wedge pressure |
| PDE5 | Phosphodiesterase-5 |
| PH | Pulmonary hypertension |
| PPS | Per-Protocol Set |
| PVR | Pulmonary vascular resistance |
| RHC | Right heart catheterization |
| RRT | Renal replacement therapy |
| RV | Right ventricle |
| RVAD | Right ventricular assist device |
| RVF | Right ventricular failure |
| RVFAC | Right ventricular fractional area change |
| RVLS | Global RV longitudinal strain |
| RVSI | Right ventricular sphericity index |
| s.c. | Subcutaneous |
| S′ | Tricuspid peak annular velocity s′ |
| SAE | Serious adverse event |
| SAP | Statistical analysis plan |
| SBP | Systolic blood pressure |
| SD | Standard deviation |
| SOC | System organ class |
| SS | Safety Set |
| ST2 | Member of the interleukin 1 receptor family |
| SUSAR | Suspected unexpected serious adverse reaction |
| SVO₂ | Mixed venous oxygen saturation |
| TAH | Total artificial heart |
| TAPSE | Tricuspid annular plane systolic excursion |
| TNF-α | Tumor necrosis factor-α |
| TPR | Total pulmonary resistance |
| ULN | Upper limit of the normal range |
| WHO | World Health Organization |
| WU | Wood unit |

### Example 1

This is a double-blind, placebo-controlled, multicenter, parallel-group Phase 2 study assessing the efficacy and safety of macitentan in PH post-LVAD implantation. Approximately 57 adult patients with PH post-LVAD implantation were randomized (1:1) to receive either macitentan 10 mg, or matching placebo, once daily orally.

After implantation of the LVAD, patients meet baseline hemodynamic criteria of PH via RHC during the last measurement prior to the first dose of drug, defined as mPAP≥25 mmHg at rest and PAWP≤18 mmHg and PVR>3 WU. Patients are randomized within 90 days of surgical implantation of LVAD and within 14 days of Baseline RHC.

Patients are considered stable for 48 h prior to the Baseline RHC. Stability is defined as having no LVAD pump speed/flow rate changes, being on a stable dose of oral diuretics, receiving no i.v. inotropes or vasopressors, and being able to ambulate, for 48 h prior to the Baseline RHC.

The periods include the following. See, Table 3.

### Screening Period (Visit 1)

All Screening assessments are performed within the 90-day Screening period, beginning with LVAD implantation and prior to Randomization. Screening and Randomization can occur on the same day, provided that all procedures of Visit 1 are completed before the procedures of Visit 2 are initiated. Visit 1 includes:
Obtain patient number.
Recording of demographics, Baseline characteristics, and medical history.
Recording of previous and concomitant medications.
Physical examination.
Measurement of vital signs, height and body weight.
Complete laboratory tests, including pregnancy test for women of childbearing potential.
Recording of methods of contraception (for females of childbearing potential only) and initiation of protocol-compliant contraception if applicable.
RHC (unless the procedure was already performed and would be within 90 days of Randomization).
Recording of AEs and SAEs.

### Randomization (Visit 2)

Visit 2 corresponds to the start of the treatment period (Day 1) for patients that are eligible after all Screening assessments have been performed. It is within 90 days after LVAD implantation and within 14 days of Baseline RHC. Visit 2 includes:
Recording of changes in concomitant medications.
Measurement of vital signs and body weight.
Complete laboratory tests, including serum pregnancy test for women of childbearing potential.
Assessment of methods of contraception (for females of childbearing potential only).
Blood sample for serum NT-proBNP.
Blood sample for additional circulating biomarkers such as: IL-6, TNF-α, hsCRP, ET-1, galectin-3, GDF-15, interleukin 1 receptor ST2, NGAL, copeptin, hs-cTnT, Cystatin-C and osteopontin.
WHO FC.
Echo (i.e., 3D, 2D, M-mode and Doppler echocardiography) is performed. Those include: TAPSE, S′, RVFAC, E′, A′, global RVLS, RV area and RVSI. Additionally, right and left ventricular echo variables may be analyzed in an exploratory format (post-hoc) based on available scientific evidence. Baseline ECHO occurs on the day of Randomization.
Recording of AEs/SAEs.

Treatment period: Treatment is defined as the dosing (intake) by the patient of double-blind drug (macitentan or placebo). The treatment period starts immediately after Randomization with the first dose of drug at the end of Visit 2 (Day 1) and ends with EOT on the day of the last dose of drug (scheduled Day 84, Week 12), or earlier in case of premature discontinuation of treatment. The schedule of visits during the treatment period includes:

### Week 4 (Visit 3)

Visit 3 is scheduled 4 weeks (±7 days) after Randomization. Visit 3 includes:
Recording of changes in concomitant medications.
Complete laboratory tests, including serum pregnancy test.
Assessment of methods of contraception.
Recording of AEs and SAEs.

### Week 8 (Visit 4)

Visit 4 is scheduled 8 weeks (±7 days) after Randomization. Visit 4 includes:
Recording of changes in concomitant medications.
Complete laboratory tests, including serum pregnancy test for women of childbearing potential.
Assessment of methods of contraception (for females of childbearing potential only).
Recording of AEs and SAEs.

### End-of-Treatment (Visit 5)

EOT is scheduled 12 weeks (±7 days) after Randomization, or earlier in case of premature discontinuation of treatment. EOT includes:
Recording of changes in concomitant medications.
Physical examination.
Measurement of vital signs and body weight.
Complete laboratory tests, including serum pregnancy test for women of childbearing potential.
Assessment of methods of contraception.
RHC.
Blood sample for serum NT-proBNP.
Blood sample for additional circulating biomarkers such as: IL-6, TNF-α, hsCRP, ET-1, galectin-3, GDF-15, interleukin 1 receptor ST2, NGAL, copeptin, hs-cTnT, Cystatin-C and osteopontin.
WHO FC.
Echo (i.e., 3D, 2D, M-mode and Doppler echocardiography) is performed. Those include: TAPSE, S′, RVFAC, E′, A′, global RVLS, RV area and RVSI. Additionally right and left ventricular echo variables may be analyzed.
Recording of AEs and SAEs.

### End-of-Study (Visit 6)

Patients completing the treatment as planned, i.e., full 12-week period and those prematurely discontinuing treatment will enter a 30-day safety follow-up period, which ends with the EOS visit at least 30 days (±7 days) after the permanent discontinuation of treatment (last dose of drug). EOS includes:
Recording of changes in concomitant medications.
Complete laboratory tests, including serum pregnancy test for women of childbearing potential.
Assessment of methods of contraception.
Recording of AEs and SAEs.

TABLE 3
Visit and assessment schedule

| | Periods | | | | | | |
|---|---|---|---|---|---|---|---|
| | Name | | | | | | |
| | Screening | Double-Blind Treatment | | | | | Follow-up |
| | Duration | | | | | | |
| | Up to 90 | | | | | | |
| | days | 12 Weeks | | | | | 30 days |
| | Visits | | | | | | |
| | Number | | | | | | |
| | 1 | 2 | 3 | 4 | 5 | U1, U2, etc. | 6 |
| | Name | | | | | | |
| | | | | | Week 12 or | Unscheduled | |
| | Screening^{1a} | Randomization^{1b} | Week 4 | Week 8 | EOT² | Visit³ | EOS⁴ |
| | Time | | | | | | |
| | | | | | | | 30 days |
| | | | | | | Any day | (+7 days) |
| | Day −90 | | Day 28 | Day 56 | Day 84 | between | after last |
| | to Day −1 | Day 1 | (±7 days) | (±7 days) | (±7 days) | Days 1 and 84 | dose |
| Informed Consent | X | | | | | | |
| Eligibility | X | | | | | | |
| Randomization | | X | | | | | |
| Demographics & | X | | | | | | |
| baseline | | | | | | | |
| characteristics | | | | | | | |
| Medical history¹⁴ | X | | | | | | |
| Medications | X | X | X | X | X | X | X |
| Physical | X | | | | X | X | |
| examination⁵ | | | | | | | |
| Vital signs, body | X | X | | | X | X | |
| weight, height and | | | | | | | |
| BMI⁶ | | | | | | | |
| Screening | X^{7a} | X^{7a} | | | | | |
| laboratory tests | | | | | | | |
| Central laboratory | | X^{7b} | X | X | X | X | X |
| tests | | | | | | | |
| Contraception | X | X | X | X | X | X | X |
| check & Pregnancy | | | | | | | |
| test⁸ | | | | | | | |
| RHC by the | X^{9a} | | | | X^{9b} | X^{9c} | |
| thermodilution | | | | | | | |
| method | | | | | | | |
| Echocardiography | | X¹⁰ | | | X | X¹¹ | |
| NT-proBNP | | X | | | X | X | |
| WHO functional | | X | | | X | X | |
| class | | | | | | | |
| Study treatment | | X | X | X | X¹² | X | |
| dispensing/return | | | | | | | |
| AEs and SAEs¹³ | X | X | X | X | X | X | X |

^{1a}Written Informed Consent is obtained prior to initiation of any study-mandated procedures. Screening and Randomization visits may be conducted on the same day, provided eligibility is confirmed and all study-mandated procedures are completed prior to Randomization.
^{1b}Randomization is within 14 days of Baseline RHC. Study treatment starts immediately after Randomization.
²EOT occurs on the day of the last dose of study treatment or within 7 days of study treatment discontinuation if prior to Week 12 (Day 84).
³Unscheduled Visits can be performed at any time during the study, as necessary. Study specific procedure/assessments that are marked may be performed during an Unscheduled Visit.
⁴Patients completing the study as planned, i.e., full 12-week study period and those prematurely discontinuing study treatment will enter a 30-day safety follow-up period, which ends with the EOS visit at least 30 days after the permanent discontinuation of study treatment.
⁵Clinically relevant findings at the time of signing of Informed Consent are recorded.
⁶Height is only measured at Screening (in cm).
^{7a}At the time of Screening, laboratory results may be used for determination of patient status (e.g., guiding RHC decision-making). At the time of Randomization, laboratory results are used to determine patient eligibility.
^{7b}Central lab results are submitted once eligibility is confirmed and prior to Randomization. Laboratory testing includes hematology and blood chemistry. Additionally, NT proBNP, biomarkers and calculated GFR are performed on Visits 2 and 5.
⁸For women of childbearing potential only. Randomization (Visit 2) pregnancy test result are negative prior to Randomization.
^{9a}The last hemodynamic measurements after LVAD implantation and prior to the first dose of study treatment. Randomization is within 14 days of Baseline RHC.
^{9b}Repeated at Week 12 or within 7 days of permanent discontinuation of study treatment if prior to Week 12. All RHC hemodynamics are obtained by the thermodilution method.
^{9c}RHC may be performed at an Unscheduled Visit. If the Unscheduled RHC is the last RHC, the data may be entered as Week 12 or EOT RHC (see 9^{b}). If the Unscheduled RHC is before the last (Week 12 or EOT) RHC, the data may be entered as Unscheduled Visit.
¹⁰Baseline ECHO occurs on the day of Randomization.
¹¹ECHO may be performed at an Unscheduled Visit.
¹²If study treatment discontinuation occurs prior to Week 12 (Day 84), record drug return as per standard procedure.
¹³All AEs and SAEs that occur after signing the Informed Consent Form and up to 30 days after study treatment discontinuation.
¹⁴Medical history includes chronic medical conditions and new acute medical conditions within the past 6 months (e.g., anemia, hepatitis C infection, HIV infection, obstructive or restrictive lung disease, hypertension, renal disease) and any previous life-threatening conditions (e.g., myocardial infarction).

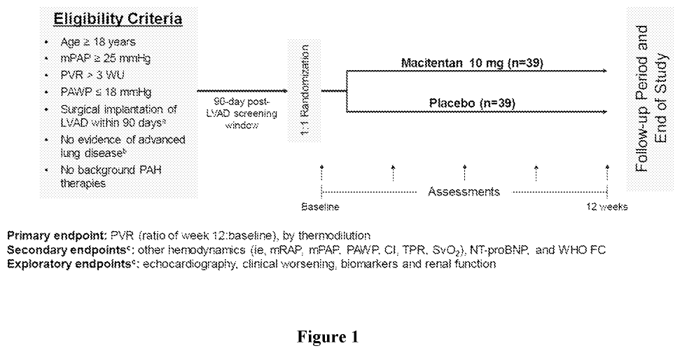

For an individual patient, the treatment ended once the EOT has been performed and the 30-day safety follow-up is complete (EOS). Patient participation is up to 90 days (Screening period)+12 weeks (double-blinded treatment period)+30 days (safety follow-up period). The overall design is depicted in FIG. 1.

### 1. Objectives

Primary objective: To evaluate the effect of macitentan 10 mg on PVR as compared to placebo in patients with PH after LVAD implantation.

### Secondary Objectives

To evaluate the effect of macitentan 10 mg as compared to placebo on cardio-pulmonary hemodynamics and disease severity in patients with PH after LVAD implantation.
To evaluate the safety and tolerability of macitentan 10 mg in patients with PH after LVAD implantation.

### Exploratory Objectives

To explore the potential effect of macitentan 10 mg as compared to placebo on right ventricular function in patients with PH after LVAD implantation.
To explore the potential effect of macitentan 10 mg as compared to placebo on selected clinical events in patients with PH after LVAD implantation.
To explore the potential effect of macitentan 10 mg as compared to placebo on renal function as measured by GFR in patients with PH after LVAD implantation.

### 2. Patient Population

Patients are male or female aged 18 years and over, and meet the hemodynamic criteria of PH post-LVAD implantation. Patients are considered clinically stable. The demographics and baseline characteristics of the patients are detailed in Tables 4 and 5.

TABLE 4
Patient Demographics

| | | |
|---|---|---|
| Macitentan | Placebo | Total |
| 10 mg (N = 28) | (N = 29) | (N = 57) |

Gender [n (%)]

| | | | | | | |
|---|---|---|---|---|---|---|
| Female | 6 | (21.4) | 6 | (20.7) | 12 | (21.1) |
| Male | 22 | (78.6) | 23 | (79.3) | 45 | (78.9) |

Age (years)

| n | 28 | | 29 | | 57 | |
|---|---|---|---|---|---|---|
| Mean (std) | 56.5 | (8.2) | 58.2 | (7.0) | 57.4 | (7.6) |

| | | | |
|---|---|---|---|
| 95% CI | 53.4, 59.7 | 55.6, 60.9 | 55.4, 59.4 |
| Median | 57.0 | 60.0 | 58.0 |
| Q1, Q3 | 53.0, 62.0 | 55.0, 63.0 | 54.0, 63.0 |
| Min, Max | 35.0, 71.0 | 41.0, 69.0 | 35.0, 71.0 |

Age Group [n (%)]

| | | | | | | |
|---|---|---|---|---|---|---|
| <65 years | 24 | (85.7) | 24 | (82.8) | 48 | (84.2) |
| ≥65 years-<75 years | 4 | (14.3) | 5 | (17.2) | 9 | (15.8) |

| | | | |
|---|---|---|---|
| ≥75 years | 0 | 0 | 0 |

Race [n (%)]

| | | | | | | |
|---|---|---|---|---|---|---|
| White | 16 | (57.1) | 18 | (62.1) | 34 | (59.6) |
| Black or African | 8 | (28.6) | 11 | (37.9) | 19 | (33.3) |
| American | | | | | | |

| | | | | | |
|---|---|---|---|---|---|
| Asian | 2 | (7.1) | 0 | 2 | (3.5) |
| Other | 2 | (7.1) | 0 | 2 | (3.5) |

Ethnicity [n (%)]

| | | | | | | |
|---|---|---|---|---|---|---|
| Hispanic or Latino | 6 | (21.4) | 1 | (3.4) | 7 | (12.3) |
| Not Hispanic or Latino | 22 | (78.6) | 28 | (96.6) | 50 | (87.7) |

TABLE 5
Baseline Characteristics (FAS)

| | | |
|---|---|---|
| Macitentan | Placebo | Total |
| 10 mg (N = 28) | (N = 29) | (N = 57) |

Weight (kg)

| n | 28 | | 29 | | 57 | |
|---|---|---|---|---|---|---|
| Mean (std) | 83.4 | (19.9) | 86.1 | (20.4) | 84.8 | (20.0) |

| | | | |
|---|---|---|---|
| Median | 77.9 | 84.5 | 80.4 |
| Q1, Q3 | 70.9, 97.7 | 74.6, 97.5 | 70.9, 97.5 |

Height (cm)

| n | 28 | | 29 | | 57 | |
|---|---|---|---|---|---|---|
| Mean (std) | 172.0 | (10.8) | 175.8 | (10.1) | 173.9 | (10.6) |

| | | | |
|---|---|---|---|
| Median | 171.4 | 175.3 | 172.7 |
| Q1, Q3 | 164.4, 178.3 | 170.2, 182.9 | 167.6, 180.3 |

BMI (kg/m²)

| n | 28 | | 29 | | 57 | |
|---|---|---|---|---|---|---|
| Mean (std) | 28.0 | (5.3) | 27.7 | (5.5) | 27.9 | (5.4) |

| | | | |
|---|---|---|---|
| Median | 27 | 27.1 | 27.1 |
| Q1, Q3 | 24.3, 30.7 | 24.5, 31.5 | 24.5, 31.0 |

Baseline Functional Class

| | | | | | | |
|---|---|---|---|---|---|---|
| CLASS I | 2 | (7.1%) | 2 | (6.9%) | 4 | (7.0%) |
| CLASS II | 7 | (25.0%) | 10 | (34.5%) | 17 | (29.8%) |
| CLASS III | 17 | (60.7%) | 16 | (55.2%) | 33 | (57.9%) |
| CLASS IV | 1 | (3.6%) | 1 | (3.4%) | 2 | (3.5%) |

| | | | | | |
|---|---|---|---|---|---|
| Missing | 1 | (3.6%) | 0 | 1 | (1.8%) |

Time from LVAD implantation to treatment (days)

| n | 28 | | 29 | | 57 | |
|---|---|---|---|---|---|---|
| Mean (std) | 44.9 | (24.7) | 46.8 | (22.9) | 45.9 | (23.6) |

| | | | |
|---|---|---|---|
| Median | 38 | 43 | 41 |
| Q1, Q3 | 24.0, 66.0 | 29.0, 60.0 | 26.0, 61.0 |

Baseline LVAD Flow rate (L/min)

| n | 28 | | 27 | | 55 | |
|---|---|---|---|---|---|---|
| Mean (std) | 4.5 | (0.9) | 4.6 | (1.1) | 4.5 | (1.0) |

| | | | |
|---|---|---|---|
| Median | 4.4 | 4.3 | 4.3 |
| Q1, Q3 | 4.1, 5.0 | 3.8, 5.5 | 4.0, 5.0 |

Baseline LVAD Power (watts)

| n | 26 | | 27 | | 53 | |
|---|---|---|---|---|---|---|
| Mean (std) | 4.4 | (0.8) | 4.3 | (0.9) | 4.3 | (0.8) |

| | | | |
|---|---|---|---|
| Median | 4.1 | 4.2 | 4.1 |
| Q1, Q3 | 3.8, 4.9 | 3.7, 4.8 | 3.8, 4.8 |

NT-proBNP (pmol/L)

| n | 27 | | 29 | | 56 | |
|---|---|---|---|---|---|---|
| Mean (std) | 2600.3 | (2079.5) | 2708.9 | (2521.6) | 2656.5 | (2298.7) |

| | | | |
|---|---|---|---|
| Median | 1943.0 | 2148.0 | 2045.5 |
| Q1, Q3 | 1257.0, 3377.0 | 1129.0, 3041.0 | 1139.5, 3098.0 |

Baseline hemoglobin (g/dL)

| n | 28 | | 28 | | 56 | |
|---|---|---|---|---|---|---|
| Mean (std) | 9.9 | (1.4) | 10.0 | (1.5) | 9.9 | (1.4) |

| | | | |
|---|---|---|---|
| Median | 9.7 | 9.5 | 9.6 |
| Q1, Q3 | 8.7, 11.0 | 8.8, 11.0 | 8.7, 11.0 |

Baseline SvO₂ (%)

| n | 28 | | 29 | | 57 | |
|---|---|---|---|---|---|---|
| Mean (std) | 56.0 | (14.5) | 55.6 | (9.0) | 55.8 | (11.9) |

| | | | |
|---|---|---|---|
| Median | 56.5 | 54 | 55 |
| Q1, Q3 | 47.5, 62.6 | 50.0, 62.0 | 49.0, 62.1 |

Baseline Kussmaul Sign [n (%)]

| | | | | | | |
|---|---|---|---|---|---|---|
| Positive | 2 | (7.1%) | 2 | (6.9%) | 4 | (7.0%) |
| Negative | 21 | (75.0%) | 19 | (65.5%) | 0 | (70.2%) |
| Missing | 5 | (17.9%) | 8 | (27.6%) | 13 | (22.8%) |

Baseline Right atrial oxygen saturation (%)

| N | 23 | | 18 | | 41 | |
|---|---|---|---|---|---|---|
| Mean (std) | 59.9 | (17.9) | 55.2 | (13.4) | 57.9 | (16.1) |

| | | | |
|---|---|---|---|
| Median | 57.00 | 51.35 | 55.10 |
| Q1, Q3 | 53.4, 67.5 | 47.0, 61.9 | 49.0, 66.0 |

Baseline mRAP (mmHg)

| n | 28 | | 29 | | 57 | |
|---|---|---|---|---|---|---|
| Mean (std) | 11.3 | (5.3) | 10.9 | (4.2) | 11.1 | (4.7) |

| | | | |
|---|---|---|---|
| Median | 11.0 | 11.0 | 11.0 |
| Q1, Q3 | 7.0, 15.5 | 8.0, 14.0 | 7.0, 15.0 |

Baseline mPAP (mmHg) (end-expiratory mean line)

| n | 28 | | 29 | | 57 | |
|---|---|---|---|---|---|---|
| Mean (std) | 31.1 | (5.1) | 31.0 | (5.8) | 31.1 | (5.4) |

| | | | |
|---|---|---|---|
| Median | 30.5 | 29.0 | 30.0 |
| Q1, Q3 | 27.5, 33.5 | 27.0, 33.0 | 27.0, 33.0 |

Baseline PAWP (mmHg) (end-expiratory mean line)

| n | 28 | | 29 | | 57 | |
|---|---|---|---|---|---|---|
| Mean (std) | 11.8 | (4.3) | 12.2 | (3.6) | 12.0 | (3.9) |

| | | | |
|---|---|---|---|
| Median | 12.5 | 13.0 | 13.0 |
| Q1, Q3 | 8.0, 15.5 | 10.0, 14.0 | 9.0, 14.0 |

Baseline Thermodilution CO (L/min)

| N | 27 | | 28 | | 55 | |
|---|---|---|---|---|---|---|
| Mean (std) | 4.6 | (1.0) | 4.4 | (0.8) | 4.5 | (0.9) |

| | | | |
|---|---|---|---|
| Median | 4.5 | 4.5 | 4.5 |
| Q1, Q3 | 3.7, 5.2 | 3.8, 4.9 | 3.7, 5.0 |

Baseline Thermodilution PVR (WU)

| n | 27 | | 28 | | 55 | |
|---|---|---|---|---|---|---|
| Mean (std) | 4.3 | (0.9) | 4.3 | (1.3) | 4.3 | (1.1) |

| | | | |
|---|---|---|---|
| Median | 4.1 | 4.2 | 4.2 |
| Q1, Q3 | 3.6, 5.0 | 3.4, 4.8 | 3.4, 5.0 |

The primary objective was to demonstrate a reduction in PVR versus Baseline with macitentan treatment as compared to placebo. Thus, patients presented with elevated PVR (>3 WU) and PAWP≤18 mmHg to rule out that their elevated PVR and mPAP is due to left-sided dysfunction. The absence of restriction on WHO FC and 6 MWD permitted patients with severely symptomatic PH to be enrolled.

Patients with hemoglobin <8.5 g/dL, or AST and/or ALT>3×ULN, and do not recover during the Screening period are excluded.

### A. Inclusion Criteria

For inclusion, the following are the inclusion criteria.
1. Written Informed Consent prior to initiation.
2. Males or females ≥18 years of age.
3. Surgical implantation of LVAD within 90 days prior to Randomization.
4. Hemodynamic evidence of PH on Baseline RHC by the thermodilution method. Baseline RHC is defined as the last hemodynamic measurements after LVAD implantation and prior the first dose of treatment. PH is defined as:
   a. mPAP≥25 mmHg and
   b. PAWP≤18 mmHg and
   c. PVR>3 WU.
5. Stabilization of the patient for 48 h prior to the Baseline RHC, defined as:
   a. No LVAD pump speed/flow rate changes and
   b. Stable dose of oral diuretics and
   c. No i.v. inotropes or vasopressors and
   d. Patient able to ambulate.
6. A woman of childbearing potential is eligible only if the following applies:
   a. A negative serum pregnancy test result during the Screening period (Visit 1) and Randomization (Visit 2) and
   b. Agreement to undertake monthly serum pregnancy tests during the treatment and up to 30 days after treatment discontinuation and
   c. Agreement to use one of the methods of contraception/follow the contraception scheme from Screening and up to at least 30 days after treatment discontinuation.
7. Patient is randomized within 14 days of Baseline RHC.

### B. Exclusion Criteria

Patients do not fulfill any of the following exclusion criteria.
1. Documented severe obstructive lung disease defined as: FEV₁/FVC<0.7 associated with FEV₁<50% of predicted value after bronchodilator administration.
2. Documented moderate to severe restrictive lung disease defined as: total lung capacity <60% of predicted value.
3. Documented pulmonary veno-occlusive disease.
4. Patients undergoing dialysis.
5. Hemoglobin <8.5 g/dL at Randomization.
6. AST or ALT>3×ULN at Randomization.
7. Severe hepatic impairment, e.g., Child-Pugh Class C liver disease.
8. Body weight <40 kg at Randomization.
9. Doppler mean blood pressure <65 mmHg at Randomization.
10. GFR<30 mL/min at Randomization.
11. Pregnant, planning to become pregnant, or breastfeeding.
12. Treatment with ERAs, PDE5 inhibitors, i.v., s.c. or oral prostanoids, or guanylate cyclase stimulators within 7 days prior to Baseline RHC or treatment initiation.
13. Treatment with inhaled prostanoids (e.g., iloprost, epoprostenol) or nitric oxide within 24 h prior to Baseline RHC or treatment initiation.
14. Treatment with strong inducers of CYP3A4 within 28 days prior to treatment initiation (e.g., carbamazepine, rifampicin, rifabutin, phenytoin and St. John's Wort).
15. Treatment with strong inhibitors of CYP3A4 within 28 days prior to initiation (e.g., ketoconazole, itraconazole, voriconazole, clarithromycin, telithromycin, nefazodone, ritonavir, saquinavir, boceprevir, telaprevir, iopinavir, fosamprenavir, darunavir, tipranavir, atazanavir, nelfinavir, amprenavir, and idinavir).
16. Treatment with another investigational drug (planned, or taken) within 28 days prior to treatment initiation.
17. Known hypersensitivity to ERAs, or to any of the treatment excipients.
18. Any condition that prevents compliance with the protocol or adherence to therapy.
19. Known concomitant life-threatening disease with a life expectancy <12 months.

### C. Criteria for Women of Childbearing Potential

A woman is considered of childbearing potential unless she meets at least one of the following criteria:
Previous bilateral salpingectomy, bilateral salpingo-oophorectomy or hysterectomy.
Premature ovarian failure confirmed by a specialist.
Pre-pubescence, XY genotype, Turner syndrome, uterine agenesis.
Post-menopausal, defined as 12 consecutive months with no menses without an alternative medical cause (ICH M3 definition).

### 3. Treatment Protocol

Treatment is double-blind, and comprises the active drug macitentan or matching placebo administered orally once daily. Up- or down-titration does not apply. Matching placebo is administered orally and also once daily.

The first administration of drug will take place during Randomization (Visit 2). Thereafter, one tablet (macitentan 10 mg or matching placebo) is taken orally every morning irrespective of food intake. If a morning dose is missed, the next dose is taken at the next scheduled morning time point (i.e., do not take one tablet in the evening and then one tablet the next morning). Two tablets are never taken on the same day.

At all subsequent visits, drug is taken before any procedures are performed. If a dose is not taken in the morning prior to arrival for the clinic visit, the next dose can be administered at the time of the clinic visit and prior to any assessments.

### A. Treatment Assignment

Eligible patients are randomized in a 1:1 ratio to either macitentan 10 mg or matching placebo. See, Table 6 for patients' exposure to the treatments.

TABLE 6
Exposure to Treatments (SS)

| | | |
|---|---|---|
| Macitentan | Placebo | Total |
| 10 mg (N = 28) | (*N = 29) | (N = 57) |

Duration of therapy (days)

| n | 28 | | 29 | | 57 | |
|---|---|---|---|---|---|---|
| Mean (std) | 79.9 | (20.7) | 80.2 | (15.1) | 80.1 | (17.9) |

| | | | |
|---|---|---|---|
| Median | 85.5 | 85.0 | 85.0 |
| Q1, Q3 | 81.5, 90.0 | 80.0, 87.0 | 80.0, 88.0 |

Total exposure (mg)

| n | 28 | | 29 | | 57 | |
|---|---|---|---|---|---|---|
| Mean (std) | 774.3 | (205.8) | 787.6 | (165.4) | 781.1 | (184.8) |

| | | | |
|---|---|---|---|
| Median | 830.0 | 830.0 | 830.0 |
| Q1, Q3 | 755.0, 890.0 | 760.0, 870.0 | 760.0, 880.0 |

Compliance [n (%)]

| | | | | | | |
|---|---|---|---|---|---|---|
| <80% | 1 | (3.6%) | 1 | (3.4%) | 2 | (3.5%) |
| ≥80% and ≤120% | 27 | (96.4%) | 28 | (96.6%) | 55 | (96.5%) |

Average daily dose (mg/day)

| n | 28 | | 29 | | 57 | |
|---|---|---|---|---|---|---|
| Mean (std) | 9.707 | (0.6040) | 9.775 | (0.5975) | 9.742 | (0.5963) |
| Median | 9.832 | | 9.889 | | 9.877 | |
| Q1, Q3 | 9.558, 10.056 | | 9.615, 10.000 | | 9.570, 10.000 | |
| Subject-year of exposure [n* (%)] | 28 | (100) | 29 | (100) | 57 | (100) |
| Number of patients × mean | 6.1 | | 6.4 | | 12.5 | |
| exposure (years) | | | | | | |

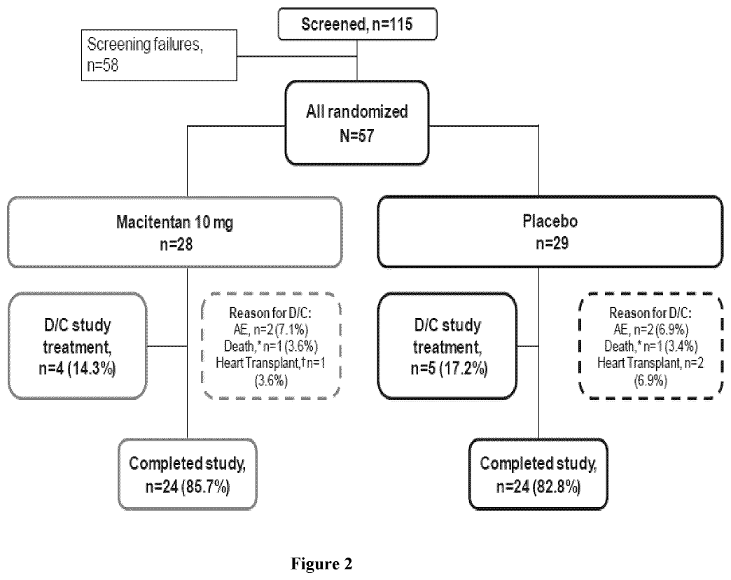

At Visit 1 (Screening), all screened patients are assigned a number. At Visit 2 (Randomization), the patient is randomized. This is performed in a double-blind fashion and unblinding is performed for final analyses. When a SUSAR occurs for a patient participating, unblinding of the treatment assignment is unblinded. SUSARs are considered in a blinded fashion. See, FIG. 2 for the disposition of patients.

Criteria for interruption/premature discontinuation of treatment include:
(i) Pregnancy: If a female patient becomes pregnant while on treatment, treatment is discontinued immediately.
(ii) Liver aminotransferases abnormalities: For abnormal laboratory results early in the Screening period (e.g., high ALT or AST), the patient may be monitored, provided with appropriate treatment, and reassessed throughout the Screening period (90 days from LVAD implantation) for eligibility.

### Interruption of Treatment

Treatment is interrupted in the following case: aminotransferases (i.e., ALT and/or AST)≥3 and ≤8×ULN

In such a case, a re-test of aminotransferases (ALT and AST), total and direct bilirubin, and alkaline phosphatase is performed within one week. If AST and/or ALT elevation is confirmed, aminotransferases, total and direct bilirubin, and alkaline phosphatase levels are monitored weekly until values return to pre-treatment levels, or within normal ranges. If the aminotransferase values return to pre-treatment levels or within normal ranges, reintroduction of treatment can be considered. Interruptions are for less than 2 consecutive weeks; longer interruptions lead to permanent discontinuation of treatment.

Reintroduction of treatment after treatment interruption should only be considered if the potential benefits of treatment with macitentan outweigh the potential risks, and when liver aminotransferase values are within pre-treatment levels or within normal ranges.

Liver aminotransferase levels are then checked within 3 days after reintroduction, again after a further 2 weeks, and thereafter according to the recommendations above (i.e., at monthly intervals).

Permanent discontinuation of treatment: Treatment is stopped and its reintroduction is not considered in the following cases:
Aminotransferases >8×ULN
Aminotransferases ≥3×ULN and associated clinical symptoms of liver injury, e.g., nausea, vomiting, fever, abdominal pain, jaundice, unusual lethargy or fatigue, flu like syndrome (arthralgia, myalgia, fever)
Aminotransferases ≥3×ULN and associated increase in total bilirubin ≥2×ULN.
(iii) Hemoglobin abnormalities

For abnormal results early in the Screening period (e.g. low hemoglobin), the patient may be monitored, appropriate treatment provided, and reassessed throughout the Screening period (90 days from LVAD implantation) for eligibility. In case of hemoglobin decrease from Baseline of >2.0 g/dL, a re-test is performed within 10 days, with additional laboratory evaluations that may include, but are not limited to red blood cell cellular indices (mean corpuscular volume, mean corpuscular hemoglobin, mean corpuscular hemoglobin concentration), peripheral blood smear, reticulocyte count, iron status (iron level, serum ferritin, total iron binding capacity, transferrin saturation), lactate dehydrogenase, indirect bilirubin.

Treatment may be temporarily interrupted in any of the following situations:
A decrease in hemoglobin to <6.5 g/dL,
A decrease in hemoglobin from Baseline of >5.0 g/dL (baseline hemoglobin: last value obtained prior to first dose of treatment.
The need for transfusion.
(iv) Initiation of a prohibited concomitant medication: Treatment is permanently discontinued if a prohibited concomitant medication is initiated.
(v) Clinical evidence of worsening PH or HF: Treatment may be discontinued if the patient demonstrates signs and symptoms of progressive HF reflecting worsening PH and RVF. Examples include, but are not limited to, the need for RV support pharmacologically with inotropes or mechanically with the implantation of RVAD, the development of diuretics resistance, objective evidence of declining RV function on echocardiography, evidence of increased PVR and/or decreased CO on RHC, and the need for escalation of PH therapies.
(vi) Cardiac transplantation: Treatment is permanently discontinued if a patient becomes a recipient of a cardiac transplant. RHC obtained prior to cardiac transplantation, to define the patient candidacy, is captured as EOT RHC. In case of premature treatment discontinuation, all EOT assessments, especially RHC, may be performed as detailed per EOT visit.

### B. Previous and Concomitant Therapy

A previous therapy is any treatment for which the end date is prior to the signing of informed consent. A therapy that is concomitant is any treatment for which the start date is on or after the signing of informed consent or up to 30 days after treatment discontinuation. A therapy that is treatment-concomitant is any treatment for which the start date is on or after the first administration of treatment up to Week 12 or earlier in case of premature discontinuation of treatment.

### Allowed Concomitant Therapy

Oral or i.v. diuretics are allowed and may be adjusted during the treatment period. Treatment with oral diuretics is allowed if ongoing at a stable dose for at least 48 h prior to the Baseline RHC.

### Prohibited Concomitant Therapy

PAH-specific therapy (ERAs, i.v., s.c., inhaled or oral prostanoids, PDE5 inhibitors, guanylate cyclase stimulators).
iNO.
Strong CYP3A4 inhibitors (e.g., ketoconazole, itraconazole, voriconazole, clarithromycin, telithromycin, nefazodone, ritonavir, saquinavir, boceprevir, telaprevir, iopinavir, fosamprenavir, darunavir, tipranavir, atazanavir, nelfinavir, amprenavir, and idinavir) and strong inducers of CYP3A4 (e.g., carbamazepine, rifampicin, rifabutin, phenytoin and St. John's Wort).
Any investigational drug.

### Allowed LVAD Flow Rate

The LVAD pump speed/flow rate may be adjusted during the treatment period, and during the Baseline RHC. The LVAD pump speed/flow rate is stable for at least 48 h prior to Baseline RHC.

### 4. Endpoints

Baseline is defined as the last value obtained prior to first dose of treatment.

### A. Efficacy Endpoints

(i) Primary efficacy endpoint: The primary efficacy endpoint is PVR ratio of Week 12 to Baseline. PVR is obtained by the thermodilution method. See, Table 7 for the FAS, Table 8 for the ES, and Table 9 for the PPS.

TABLE 7
Primary endpoint PVR (week 12: Baseline) (FAS)

| | | |
|---|---|---|
| Pulmonary vascular | Macitentan | Placebo |
| resistance (PVR) | 10 mg (N = 28) | (N = 29) |

PVR at Baseline (WU)

| | | |
|---|---|---|
| n | 27 | 28 |
| Mean (std) | 4.317 (0.9338) | 4.314 (1.2772) |
| Median | 4.074 | 4.154 |
| Q1, Q3 | 3.556, 5.000 | 3.420, 4.773 |

PVR at Week 12/EOT (WU)

| | | |
|---|---|---|
| n | 24 | 25 |
| Mean (std) | 2.555 (1.0607) | 3.259 (1.3400) |
| Median | 2.644 | 3.469 |
| Q1, Q3 | 1.941, 3.190 | 2.286, 3.784 |

Ratio to Baseline

| | | |
|---|---|---|
| n | 27 | 28 |
| Mean (std) | 0.585 (0.2280) | 0.757 (0.2578) |
| Median | 0.573 | 0.745 |
| Q1, Q3 | 0.467, 0.753 | 0.548, 0.897 |

PVR (Calculated by Programming) - Primary Analysis

| | | |
|---|---|---|
| n¹ | 27 | 28 |

Comparison to Placebo

| | |
|---|---|
| Geometric Mean Ratio | 0.7393 |
| 95% confidence interval | (0.5798, 0.9426) |
| p-value | 0.0158 |
| (Geometric mean ratio − 1) × 100% | −26.1 |

PVR (Reported by INV) - Sensitivity Analysis

| | | |
|---|---|---|
| n¹ | 25 | 25 |

Comparison to Placebo

| | |
|---|---|
| Geometric Mean Ratio | 0.7386 |
| 95% confidence interval | (0.5616, 0.9716) |
| p-value | 0.0310 |
| (Geometric mean ratio − 1) × 100% | −26.1 |

TABLE 8
Primary endpoint: PVR (Week 12: Baseline)
(ES) - Sensitivity Analysis

| PVR (Calculated by | Macitentan 10 mg | Placebo |
|---|---|---|
| Programming) | (N = 28) | (N = 29) |
| n¹ | 24 | 25 |
| Comparison to Placebo | | |
| Geometric Mean Ratio | 0.7371 | |
| 95% confidence interval | (0.5601, 0.9702) | |
| p-value | 0.0303 | |
| (Geometric mean ratio − 1) × 100% | −26.3 | |

TABLE 9
Primary endpoint: PVR (Week 12: Baseline)
(PPS) - Sensitivity Analysis

| PVR (Calculated by | Macitentan 10 mg | Placebo |
|---|---|---|
| Programming) | (N = 28) | (N = 29) |
| n¹ | 14 | 17 |
| Comparison to Placebo | | |
| Geometric Mean Ratio | 0.6236 | |
| 95% confidence interval | (0.4447, 0.8743) | |
| p-value | 0.0079 | |
| (Geometric mean ratio − 1) × 100% | −37.6 | |

Only patients where a Ratio to Baseline can be calculated are summarized. An ANCOVA is performed on the log ratio, with a main effect for treatment and a covariate for log baseline. The geometric mean ratio is calculated as the exponentiated least squares mean difference between treatments. Two-sided p-value is presented (p < 0.05 is significant).

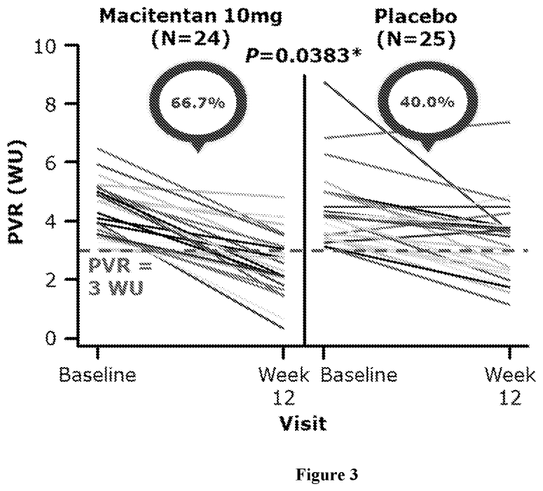

These results show that macitentan significantly reduced PVR in the early post-LVAD population. Treatment with macitentan 10 mg once daily led to a statistically significant greater reduction in PVR at Week 12 compared with placebo group, with a placebo-adjusted geometric mean ratio (GMR) of 0.74 (95% CIs: 0.58, 0.94, p=0.0158). The treatment effect (GMR-1×100%) corresponds to a statistically significant 26% reduction in PVR for macitentan vs placebo. The scatter plot in FIG. 3 shows the change in thermodilution PVR from baseline to week 12 by RHC for evaluable patients receiving either macitentan or placebo. A statistically significant greater reduction in PVR was observed in the macitentan treatment group at Week 12 compared to the placebo group

Overall, 16 (66.7%) patients in the macitentan and 10 (40.0%) patients in the placebo group had a postbaseline PVR<3 WU, which is statistically significant (based on Cochran-Mantel-Haenszel statistics adjusting for the baseline PVR group, p=0.0383), i.e., more patients receiving macitentan achieved PVR<3 WU vs placebo in a post-hoc analysis (p=0.0383). At baseline, mean PVR was similar in both treatment arms.

Among patients with baseline PVR<4 WU, the proportion with postbaseline PVR<3 WU was 90.0% in the macitentan group (n=10) and 63.6% in the placebo group (n=11). Among patients with baseline PVR>4 WU, the proportion with postbaseline PVR<3 WU was 50.0% in the macitentan group (n=14) and 21.4% in the placebo group (n=14).
(ii) Secondary efficacy endpoints

Change from Baseline to Week 12 in mRAP, mPAP, PAWP, CI, TPR, and mixed SvO₂, all measured at rest.

Change from Baseline to Week 12 in NT-proBNP.

Change from Baseline to Week 12 in WHO FC. See, Table 10.

TABLE 10
Change from baseline in WHO FC (FAS)

| | | | | Odds Ratio | |
|---|---|---|---|---|---|
| | | | | Macitentan relative | |
| Number (%) Change in | Macitentan | Placebo | | to Placebo (95% | P- |
| WHO FC | (N = 25) | (N = 25) | | Confidence Interval) | value* |
| Worsening (change > 0) | 2 (8.0) | 2 | (8.0) | 1.333 (0.417, 4.255) | 0.628 |
| No change (change = 0) | 13 (52.0) | 15 | (60.0) | | |

| | | | |
|---|---|---|---|
| Improvement (change < 0) | 10 (40.0) | 8 | (32.0) |

Higher OR indicated better change. Compared by Ordered Logistic Regression adjusting for baseline. Baseline WHO functional class is entered in the model as a categorical variable (WHO FC I/II vs. III/IV). The proportion of patients with WHO FC worsened, unchanged, and improved was compared by an ordered logistic regression, adjusting for baseline.

(iii) Exploratory efficacy endpoints
Change from Baseline to Week 12 in echocardiographic variables that include 2D global RVLS, RVSI, RV end systolic area, RV end diastolic area, TAPSE, S′, RVFAC, and E′, A′
Time to first occurrence of clinical events, from enrollment to Week 12. These clinical events include hospital admission for HF, re-initiation of i.v. diuretics ≥48 h duration, re-initiation of i.v. inotropes ≥48 h duration, initiation of PDE5 inhibitors, need for RVAD/TAH, need for RRT, or death
Days in the hospital after hospitalization for HF.
Change in GFR from Baseline to Week 12.

### B. Safety Endpoints

Treatment-emergent AEs up to 30 days after treatment discontinuation.
AEs leading to premature discontinuation of treatment.
Death up to 30 days after treatment discontinuation.
Treatment-emergent SAEs up to 30 days after treatment discontinuation.
Change from Baseline to EOT in vital signs.
Occurrence of liver function test (ALT and/or AST) abnormality (≥3×ULN; ≥3 and <5×ULN; ≥5 and <8×ULN; ≥8×ULN) up to EOT.
Occurrence of ALT and/or AST abnormality ≥3×ULN associated with bilirubin ≥2×ULN up to EOT.
Proportion of patients with treatment-emergent hemoglobin abnormality (<10.0 g/dL, and <8.0 g/dL) as compared to Baseline up to EOT.
Treatment-emergent marked laboratory abnormalities up to EOT.

### C. Biomarker Endpoints

Change in NT-proBNP is assessed from Baseline to Week 12 (EOT) as part of the secondary efficacy endpoints analysis.

### 4. Assessments

When applicable, the assessments may be performed in the following order:
Blood sampling
Physical examination
WHO FC
RHC at rest
Echo (i.e., 3D, 2D, M-mode and Doppler echocardiography)

### A. Screening/Baseline Assessments

Date of informed consent, Baseline demographics (sex, age, race, ethnicity, body weight, height) as well as a reason for why a woman is not considered of child-bearing potential (if applicable) are noted at Visit 1/Screening only after Informed Consent has been signed.

Complete, clinically relevant medical history (previous and ongoing at Randomization) and disease characteristics, vital signs, BMI, and previous/ongoing medications are documented at Visit 1/Screening. Historical data of pre-LVAD RHC and non-Baseline post-LVAD RHC may be entered.

A physical examination of the patient may be performed at Visit 1/Screening and Visit 2/Randomization.

Serum laboratory and pregnancy testing may be performed at Visit 1/Screening and repeated at Visit 2/Randomization, whereas blood sampling for Baseline NT-proBNP should only be performed at Visit 2/Randomization.

At Visit 2/Randomization, WHO FC, vital signs, and body weight is assessed.

### B. Efficacy Assessments

Eligibility may be considered for all post-LVAD patients provided they have clinical or hemodynamic evidence of PH (e.g., PVR>3 WU) on pre-LVAD RHC or immediate post-LVAD hemodynamics. Patients without clinical or hemodynamic evidence of PH should not be screened. The patient is stable for 48 h prior to the Baseline RHC. Stabilization is defined as:
No LVAD pump speed/flow rate changes, and
Stable dose of oral diuretics, and
No i.v. inotropes or vasopressors, and
Patient able to ambulate.

Baseline RHC is defined as the last hemodynamic measurements after LVAD implantation and prior to first dose of treatment. Hemodynamic evidence of PH on Baseline RHC is one of the inclusion criteria. PH is defined as:

$$mPAP \geq 25 mm Hg , PAWP \leq 18 mm Hg and PVR > 3 WU .$$

An RHC conducted prior to signing the Informed Consent may be accepted as the Baseline RHC. Baseline RHC is performed by the thermodilution method, after LVAD implantation and prior to first dose of treatment (which occurs within 90 days post-surgical implantation of LVAD). Randomization (Visit 2) is within 14 days of Baseline RHC. Patients are excluded if they were treated with ERAs, PDE5 inhibitors, i.v., s.c., or oral prostanoids, or guanylate cyclase stimulators within 7 days prior to Baseline RHC or treatment initiation. Patients are excluded if they were treated with inhaled prostanoids (e.g., iloprost, epoprostenol), nitric oxide or i.v. inotropes or vasopressors within 24 h prior to Baseline RHC or treatment initiation. LVAD pump speed/flow rate may be adjusted during the treatment period and during the Baseline RHC.

An RHC is repeated at Week 12, or within 7 days of permanent discontinuation of treatment if prior to Week 12. The Week 12 RHC is performed by the thermodilution method, and not within 24 h of treatment with i.v. inotropes or vasopressors.

The following variables also are measured: pulse rate, PAWP, mRAP, systolic/diastolic/mean PAP, CO, and SVO₂ and non-invasive mean, SBP and DBP if available. PVR is calculated. CO measurements, and PVR calculations, during both Baseline and Week 12 RHC is obtained via the thermodilution method. All primary end point calculations is based on thermodilution method-calculated PVR.

The following hemodynamic variables are calculated: PVR, TPR, and CI.

See, Table 11 for the change in baseline for the RHC parameters (mPAP, mRAP, PAWP, CO, CI, mPAP, TPR, SvO₂, DPG, and TPG).

TABLE 11
Change in Baseline in RHC Parameters (FAS)

| | | |
|---|---|---|
| | Macitentan 10 mg | Placebo |
| Parameters | (N = 28) | (N=29) |

mPAP (end-expiratory mean line), mmHg

| | | |
|---|---|---|
| n¹ | 24 | 25 |
| Least squares mean | −4.2251 | −3.9199 |
| Standard error of least squares mean | 1.6675 | 1.6337 |
| 95% CI | (−7.5835, −0.8666) | (−7.2104, −0.6294) |
| Comparison to Placebo | −0.3052 | |
| Difference of least squares mean | | |
| 95% confidence interval | (−5.0109, 4.4006) | |
| p-value | 0.8967 | |

mRAP, mmHg

| | | |
|---|---|---|
| n¹ | 24 | 25 |
| Least squares mean | 0.6222 | −1.5173 |
| Standard error of least squares mean | 0.9584 | 0.9390 |
| 95% CI | (−1.3080, 2.5524) | (−3.4085, 0.3738) |
| Comparison to Placebo | 2.1396 | |
| Difference of least squares mean | | |
| 95% confidence interval | (−0.5653, 4.8444) | |
| p-value | 0.1181 | |

Change in PAWP at Day 84 or EOT compared to Baseline (mmHg)

| | | |
|---|---|---|
| n¹ | 24 | 25 |
| Least squares mean | 3.4038 | 1.0524 |
| Standard error of least squares mean | 1.2535 | 1.2281 |
| 95% CI | (0.8791, 5.9284) | (−1.4212, 3.5260) |
| Comparison to Placebo | 2.3514 | |
| Difference of least squares mean | | |
| 95% confidence interval | (−1.1859, 5.8886) | |
| p-value | 0.1873 | |

Change in CO at Day 84 or EOT compared to Baseline (L/min)

| | | |
|---|---|---|
| n¹ | 24 | 25 |
| Least squares mean | 0.3655 | 0.1591 |
| Standard error of least squares mean | 0.1890 | 0.1852 |
| 95% CI | (−0.0152, 0.7462) | (−0.2139, 0.5321) |
| Comparison to Placebo | 0.2064 | |
| Difference of least squares mean | | |
| 95% confidence interval | (−0.3277, 0.7405) | |
| p-value | 0.4405 | |

Change in CI at Day 84 or EOT compared to Baseline (L/min/m²)

| | | |
|---|---|---|
| n¹ | 24 | 25 |
| Least squares mean | 0.1284 | 0.0074 |
| Standard error of least squares mean | 0.0824 | 0.0807 |
| 95% CI | (−0.0376, 0.2943) | (−0.1552, 0.1700) |
| Comparison to Placebo | 0.1209 | |
| Difference of least squares mean | | |
| 95% confidence interval | (−0.1129, 0.3548) | |
| p-value | 0.3032 | |

Change in mPAP at Day 84 or EOT compared to Baseline (mmHg)

| | | |
|---|---|---|
| n¹ | 24 | 25 |
| Least squares mean | −4.8854 | −3.5819 |
| Standard error of least squares mean | 1.4279 | 1.3680 |
| 95% CI | (−7.7632, −2.0075) | (−6.3389, −0.8249) |
| Comparison to Placebo | −1.3035 | |
| Difference of least squares mean | | |
| 95% confidence interval | (−5.3454, 2.7385) | |
| p-value | 0.5191 | |

Change in TPR at Day 84 or EOT compared to Baseline (mmHg min/L)

| | | |
|---|---|---|
| n¹ | 24 | 25 |
| Least squares mean | −1.4175 | −0.9754 |
| Standard error of least squares mean | 0.4195 | 0.4110 |
| 95% CI | (−2.2625, −0.5725) | (−1.8032, −0.1475) |
| Comparison to Placebo | −0.4422 | |
| Difference of least squares mean | | |
| 95% confidence interval | (−1.6271, 0.7427) | |
| p-value | 0.4562 | |

Change in SvO₂ at Day 84 or EOT compared to Baseline (%)

| | | |
|---|---|---|
| n¹ | 24 | 25 |
| Least squares mean | 4.7801 | 4.3515 |
| Standard error of least squares mean | 1.6798 | 1.6458 |
| 95% CI | (1.3967, 8.1634) | (1.0367, 7.6664) |
| Comparison to Placebo | 0.4285 | |
| Difference of least squares mean | | |
| 95% confidence interval | (−4.3118, 5.1689) | |
| p-value | 0.8563 | |

Change in DPG at Day 84 or EOT compared to Baseline (mmHg)

| | | |
|---|---|---|
| n¹ | 23 | 25 |
| Baseline, mean (SD) | 9.87 (3.035) | 9.96 (5.029) |
| Day 84 or EOT, mean (SD) | 3.87 (5.619) | 6.60 (5.339) |
| Least squares mean change | −6.0383 | −3.3248 |
| from baseline (95% CI) | (−8.0143, −4.0624) | (−5.2199, −1.4296) |
| Comparison to Placebo | −2.7136 | |
| Difference of least squares mean | | |
| 95% confidence interval | (−5.4533, 0.0262) | |
| p-value | 0.0521 | |

Change in TPG at Day 84 or EOT compared to Baseline (mmHg)

| | | |
|---|---|---|
| n¹ | 24 | 25 |
| Baseline, mean (SD) | 19.83 (3.795) | 19.60 (6.371) |
| Day 84 or EOT, mean (SD) | 12.13 (4.902) | 14.70 (5.283) |
| Least squares mean change | −7.6521 | −4.9500 |
| from baseline (95% CI) | (−9.5799, −5.7243) | (−6.8387, −3.0612) |
| Comparison to Placebo | −2.7022 | |
| Difference of least squares mean | | |
| 95% confidence interval | (−5.4035, −0.0009) | |
| p-value | 0.0499 | |

The geometric mean ratio is presented and was calculated as the exponentiated least squares mean difference between treatments. Difference of least squares mean is presented.
DPG = dPAP − PAWP;
TPG = mPAP − PAWP

These results suggest that the use of macitentan improved LV filling in patients with post-LVAD implantation. See, e.g., PAWP and CO data, among others. RHC analyses demonstrated that TPG statistically significantly decreased with macitentan vs placebo with a difference of least squares mean of −2.7 and a p value of 0.0499.

A blood sample is drawn at Randomization (Visit 2) and EOT (Visit 5) for the analysis of serum NT-proBNP. See, Table 12 for serum NT-proBNP at Week 12 compared to baseline for the FAS.

TABLE 12
NT-proBNP (Week 12: Baseline) (FAS)

| | | |
|---|---|---|
| NT-proBNP | Macitentan 10 mg (N = 28) | Placebo (N = 29) |

Baseline (ng/L)

| | | |
|---|---|---|
| n | 22 | 24 |
| Mean (std) | 2856.4 (2208.79) | 2758.9 (2746.12) |
| Median | 2218.0 | 2023.5 |
| Q1, Q3 | 1330.0, 3861.0 | 964.0, 3098.0 |

Week 12/EOT (ng/L)

| | | |
|---|---|---|
| n | 22 | 24 |
| Mean (std) | 2435.4 (2620.75) | 1559.1 (1052.68) |
| Median | 1098.0 | 1400.0 |
| Q1, Q3 | 687.0, 2788.0 | 709.5, 2404.5 |

Ratio to Baseline

| | | |
|---|---|---|
| n | 22 | 24 |
| Mean (std) | 0.8 (0.57) | 0.8 (0.53) |
| Median | 0.6 | 0.7 |
| Q1, Q3 | 0.4, 1.2 | 0.4, 1.0 |
| n¹ | 22 | 24 |
| Comparison to Placebo | 1.1086 | |
| Geometric Mean Ratio | | |
| 95% confidence interval | (0.7488, 1.6411) | |
| p-value | 0.5990 | |
| (Geometric mean | 10.9 | |
| ratio − 1) × 100% | | |

Only patients where a ratio to baseline can be calculated are summarized. NT-proBNP was analyzed using a geometric mean ratio and compared by an ANCOVA model. ANCOVA is performed on the log ratio, with a main effect for treatment and a covariate for log baseline. A two-sided p-value is presented (p < 0.05 is significant).
¹Number of patients contributing to the analysis.

Echo (i.e., 3D, 2D, M-mode and Doppler echocardiography) is performed on Visit 2/Randomization, which will represent the Baseline measurements and be repeated at the Week 12 visit. Baseline ECHO occurs on the day of Randomization. The following variables are assessed: 2D RVLS, RVSI, RV end systolic area, RV end diastolic area, TAPSE, S′RVFAC, E′, and A′.

### B. Clinical Events

Time to first occurrence of clinical events, from enrollment to Week 12 are noted. These clinical events include hospital admission for HF, re-initiation of i.v. diuretics ≥48 h duration, re-initiation of i.v. inotropes ≥48 h duration, initiation of PDE5 inhibitors, need for RVAD/TAH, need for RRT, and death

### C. Laboratory Tests

Hematology: Hematology tests are performed at every visit (Visits 1 to 6) and include Hemoglobin, hematocrit, erythrocyte count, leukocyte count with differential counts, and platelet count.

Clinical chemistry: Clinical chemistry tests are performed at every visit (Visits 1 to 6) and include AST/ALT, alkaline phosphatase, total and direct bilirubin, LDH; creatinine, BUN; uric acid; glucose; cholesterol, triglycerides; sodium, potassium, chloride, calcium; and protein, albumin.

GFR: GFR is estimated on the basis of serum creatinine levels.

Pregnancy test: A serum pregnancy test for women of childbearing potential is provided performed at Visits 1 to 6 and if pregnancy is suspected.

### D. Biomarker and Genetic Assessments

Besides NT-proBNP, additional circulating biomarkers such as IL-6, TNF-α, hsCRP, ET-1, galectin-3, GDF-15, interleukin 1 receptor ST2, NGAL, copeptin, high sensitivity cardiac troponin T hs-cTnT, Cystatin-C and osteopontin, may be analyzed.

### 6. Safety Definitions

### A. Adverse Events

An AE is any adverse change, i.e., any unfavorable and unintended sign, including an abnormal laboratory finding, symptom, or disease that occurs in a patient. AEs include:
Exacerbation of a pre-existing disease.
Increase in frequency or intensity of a pre-existing episodic disease or medical condition.
Disease or medical condition detected or diagnosed even though it may have been present prior to the start of the treatment.
Continuous persistent disease or symptoms present at treatment start that worsen following the start.
Abnormal assessments, e.g., change on physical examination, ECG findings, if they represent a clinically significant finding that was not present at treatment start or worsened during the course of the treatment.
Laboratory test abnormalities if they represent a clinically significant finding, symptomatic or not, which was not present at treatment start or worsened during the course of the treatment or led to dose reduction, interruption or permanent discontinuation of treatment.

A treatment-emergent AE is any AE temporally associated with the use of treatment (from treatment initiation until 30 days after treatment discontinuation). Overdose, misuse, and abuse of the treatment may be considered an AE.

The intensity of clinical AEs is graded on a three-point scale-mild, moderate, severe. The three categories of intensity are defined as follows:
Mild: The event may be noticeable to the patient. It does not influence daily activities, and usually does not require intervention.
Moderate: The event may make the patient uncomfortable. Performance of daily activities may be influenced, and intervention may be needed.
Severe: The event may cause noticeable discomfort, and usually interferes with daily activities. The patient may not be able to continue, and treatment or intervention is usually needed.

Each AE is assessed as to whether or not there is a reasonable possibility of causal relationship to treatment. An AE is defined as related to treatment design or protocol-mandated procedures if it appears to have a reasonable possibility of a causal relationship to either the design or to protocol-mandated procedures. Examples include discontinuation of a patient's previous treatment during a washout period leading to exacerbation of underlying disease.

Tables 13-15 provide the overall summary of adverse events (Table 11), AEs leading to treatment discontinuation for the SS (Table 12), and frequently Reported AEs for the SS in ≥10% of the treatment group

TABLE 13
Overall Summary of Adverse Events

| | | | | |
|---|---|---|---|---|
| Any Adverse Event | 26 | (92.9) | 25 | (86.2) |
| Any Severe Adverse Event | 9 | (32.1) | 9 | (31.0) |
| Any Treatment-Related Adverse Event | 6 | (21.4) | 7 | (24.1) |
| Any Events of Interest* | 10 | (35.7) | 8 | (27.6) |
| Any Severe Treatment-Related Adverse | 2 | (7.1) | 1 | (3.4) |
| Event | | | | |
| Any Adverse Event With Outcome of Death | 1 | (3.6) | 2 | (6.9) |
| Any Serious Adverse Event | 16 | (57.1) | 16 | (55.2) |
| Any Treatment-Related Serious Adverse | 2 | (7.1) | 1 | (3.4) |
| Event | | | | |
| Any Adverse Event Leading to Treatment | 4 | (14.3) | 2 | (6.9) |
| Discontinuation† | | | | |
| Any Treatment-Related Adverse Event | 2 | (7.1) | 1 | (3.4) |
| Leading to Treatment Discontinuation | | | | |

Only treatment-emergent adverse events are summarized.
*Includes SMQs for anemia, hypotension, and edema/fluid retention.
†One patient in the macitentan arm experienced cerebral haemorrhage unrelated to the drug, which was reported as discontinuation of the drug due to death; one patient in the macitentan arm experienced acute kidney injury (unrelated) and worsening right heart failure (unrelated), which were reported as discontinuation of the drug due to heart transplant.

TABLE 14
AEs Leading to Treatment Discontinuation (SS)

| | Macitentan | Placebo |
|---|---|---|
| System Organ Class | (N = 28) | (N = 29) |
| Preferred Term | n (%) | n (%) |
| Any Adverse Event Leading to Treatment | 4* (14.3) | 2 (6.9) |
| Discontinuation | | |
| Blood and lymphatic system disorders | 0 | 2 (6.9) |
| Anaemia | 0 | 1 (3.4) |
| Haemolysis | 0 | 1 (3.4) |
| Nervous system disorders | 1 (3.6) | 1 (3.4) |
| Cerebral haemorrhage | 1 (3.6) | 0 |
| Haemorrhagic stroke | 0 | 1 (3.4) |
| Cardiac disorders | 1 (3.6) | 0 |
| Right ventricular failure | 1 (3.6) | 0 |
| General disorders and administration site | 1 (3.6) | 0 |
| conditions | | |
| Oedema peripheral | 1 (3.6) | 0 |
| Investigations | 1 (3.6) | 0 |
| Blood alkaline phosphatase increased | 1 (3.6) | 0 |
| Blood bilirubin increased | 1 (3.6) | 0 |
| International normalised ratio increased | 1 (3.6) | 0 |
| Liver function test increased | 1 (3.6) | 0 |
| Renal and urinary disorders | 1 (3.6) | 0 |
| Acute kidney injury | 1 (3.6) | 0 |

This includes AEs with action taken as “drug withdrawn.”
*One patient was reported as having discontinued the drug due to death and one patient as having continued the drug due to heart transplant.

TABLE 15
Frequently Reported AEs (SS) In ≥10% of Treatment Group

| | Macitentan | | Placebo | |
|---|---|---|---|---|
| | (N = 28) | | (N = 29) | |
| Preferred Term | n (%) | | n (%) | |
| Any Adverse Event | 26 | (92.9) | 25 | (86.2) |
| Dizziness | 4 | (14.3) | 7 | (24.1) |
| Anticoagulation drug level below | 4 | (14.3) | 4 | (13.8) |
| therapeutic | | | | |
| N-terminal prohormone brain natriuretic | 4 | (14.3) | 4 | (13.8) |
| peptide increased | | | | |
| Vomiting | 2 | (7.1) | 4 | (13.8) |
| Dyspnoea | 4 | (14.3) | 1 | (3.4) |
| Fatigue | 4 | (14.3) | 1 | (3.4) |
| Oedema peripheral | 4 | (14.3) | 1 | (3.4) |
| Ventricular tachycardia | 1 | (3.6) | 4 | (13.8) |
| Acute kidney injury | 3 | (10.7) | 1 | (3.4) |
| Hypotension | 3 | (10.7) | 1 | (3.4) |

| | | | |
|---|---|---|---|
| Complication associated with device | 0 | 3 | (10.3) |

| | | | |
|---|---|---|---|
| Cystatin C increased | 3 | (10.7) | 0 |

| | | | |
|---|---|---|---|
| Dehydration | 0 | 3 | (10.3) |
| Fall | 0 | 3 | (10.3) |

| | | | |
|---|---|---|---|
| Hyperkalaemia | 3 | (10.7) | 0 |

These results show that macitentan has a favorable safety profile in these post-LVAD implantation patients. These results show that macitentan was well tolerated in the early post-LVAD population. Specifically, reported AEs were largely either associated with underlying diseases of the study population or consistent with that observed in other studies of macitentan-treated subjects with PAH.

### B. Serious Adverse Events

An SAE is defined by the ICH guidelines as any AE fulfilling at least one of the following criteria:
Fatal.
Life-threatening: refers to an event in which the patient was at risk of death at the time of the event. It does not refer to an event that hypothetically might have caused death had it been more severe.
Requiring inpatient hospitalization, or prolongation of existing hospitalization.
Resulting in persistent or significant disability or incapacity.
Congenital anomaly or birth defect.
Medically significant: refers to important medical events that may not immediately result in death, be life-threatening, or require hospitalization but may be considered SAEs when they may jeopardize the patient, and may require medical or surgical intervention to prevent one of the outcomes listed in the definitions above.

However, complications that occur during hospitalization are AEs or SAEs (for example, if a complication prolongs hospitalization).

Table 16 shows the serious AEs in ≥2 patients in either treatment group.

TABLE 16

| | Macitentan | | Placebo | |
|---|---|---|---|---|
| Preferred term | (N = 28) | | (N = 29) | |
| Any serious adverse event, n (%) | 16 | (57.1) | 16 | (55.2) |
| Anticoagulation drug level below | 3 | (10.7) | 4 | (13.8) |
| therapeutic level | | | | |
| Acute kidney injury | 2 | (7.1) | 1 | (3.4) |

| | | | |
|---|---|---|---|
| Ventricular tachycardia | 0 | 3 | (10.3) |

| | | | |
|---|---|---|---|
| Pleural effusion | 2 | (7.1) | 0 |
| Right ventricular failure | 2 | (7.1) | 0 |
| Septic shock | 2 | (7.1) | 0 |

| | | | |
|---|---|---|---|
| Complication associated with device | 0 | 2 | (6.9) |
| Dehydration | 0 | 2 | (6.9) |
| Gastrointestinal hemorrhage | 0 | 2 | (6.9) |

Overall a similar proportion of patients in each treatment group experienced a serious adverse event. See, Table 15.

### 7. Statistical Methods

The statistical analysis plan provides full details of the analyses, data displays, and algorithms that were used for data derivations.

### A. Analysis Sets

Screened Analysis Set: This analysis set includes all patients screened and received a Screening number.

Safety Set (SS): The SS includes all patients receiving at least one dose of drug in the treatment period.

Full Analysis Set (FAS): The FAS includes all patients randomized.

Modified Full Analysis Set (FAS): The modified FAS includes all patients in the FAS that have received at least one dose of treatment in the treatment period and have a Baseline and at least one post-Baseline PVR measurement.

Per-Protocol Set (PPS): The PPS comprises all patients included in the modified FAS without major protocol deviations that affect the main analysis of the primary efficacy variable.

The primary efficacy analysis is performed on the FAS based on treatment as randomized. Secondary and exploratory efficacy analyses will also be performed on the FAS. Sensitivity analyses are conducted based on the modified FAS for the primary and selected secondary efficacy endpoints. Safety analyses related to the double-blind treatment period are performed on the SS based on treatment as received. Patient listings are based on the SS, unless otherwise specified. Patient disposition is described for the Screened Analysis Set. See, Table 17.

TABLE 17

| Population | Macitentan | | Placebo | | Total | |
|---|---|---|---|---|---|---|
| SAS [n] | 28 | | 29 | | 115 | (58 SF) |
| FAS [n] | 28 | | 29 | | 57 | |
| Patients Treated [n (%)^{a}] | 28 | (100) | 29 | (100) | 57 | (100) |
| Patients Randomized but | 0 | | 0 | | 0 | |
| Not Treated [n (%)^{a}] | | | | | | |
| Patients Treated | 0 | | 0 | | 0 | |
| Incorrectly [n (%)^{a}] | | | | | | |
| Modified FAS [n (%)^{a}] | 27 | (96.4) | 28 | (96.6) | 55 | (96.5) |
| Evaluable Set [n (%)^{a}] | 24 | (85.7) | 25 | (86.2) | 49 | (86.0) |
| PPS [n (%)^{a}] | 14 | (50.0) | 17 | (58.6) | 31 | (54.4) |

| | | | |
|---|---|---|---|
| SS [n]^{b} | 28 | 29 | 57 |

^{a}Percent of FAS.
^{b}Percentages not included because patients are grouped according to treatment actually received and not randomized group for SS. Patients treated incorrectly correspond to patients with the incorrect investigational product and not dosing mistakes.

### B. Variables

The primary efficacy variable is PVR ratio of Week 12 to Baseline. PVR [WU] is calculated as (mPAP-PAWP)/CO.

Secondary efficacy variables are changes from Baseline to Week 12, for:
mRAP (mmHg)
mPAP (mmHg)
PAWP (mmHg)
CI (L/min/m²), calculated as CO/BSA where BSA (m²)=0.007184×weight^{0.425} (kg)×height^{0.725} (cm)
TPR (WU), calculated as mPAP/CO
SVO₂ (%)
NT-proBNP (pmol/L)
WHO FC (I-IV).

Exploratory efficacy variables include:
Echocardiographic variables including 2D global RVLS, RVSI, RV end systolic area, RV end diastolic area, TAPSE, S′, RVFAC, and E′, A.′
Clinical events including hospital admission for HF, re-initiation of i.v. diuretics ≥48 h duration, re-initiation of i.v. inotropes ≥48 h duration, initiation of PDE5 inhibitors, need for RVAD/TAH, need for RRT, and death.
Days in the hospital after index hospitalization for heart failure.
Change in GFR from Baseline to Week 12.

Safety variables include:
Treatment-emergent AEs up to 30 days after treatment discontinuation.
AEs leading to premature discontinuation of treatment.
Death up to 30 days after treatment discontinuation.
Treatment-emergent SAEs up to 30 days after treatment discontinuation.
Change from Baseline to EOT in vital signs.
Proportion of patients with treatment-emergent ALT and/or AST abnormalities from Baseline up to EOT, classified as:
   ≥3×ULN;
   ≥3×ULN and <5×ULN;
   ≥5×ULN and <8×ULN;
   ≥8×ULN.
Proportion of patients with ALT and/or AST abnormality ≥3×ULN in combination with total bilirubin ≥2×ULN from Baseline up to EOT.
Proportion of patients with treatment-emergent hemoglobin abnormalities as compared to Baseline up to EOT, classified as:
   <8.0 g/dL
   <10.0 g/dL.
Treatment-emergent marked laboratory abnormalities up to EOT.

The overall type I error is α=0.025 (one-sided). The type II error is set to 0.20 and the power to 80%.

### C. Analysis of the Primary Efficacy Variables

The null hypothesis is that the mean PVR ratio is the same in the macitentan and placebo groups. The alternative hypothesis is that the mean PVR ratio is lower in the macitentan group as compared to the placebo group.

If PVR cannot be calculated due to missing PAWP, but mPAP and CO are available for the same visit, one of the following is applied:
1. If PAWP is missing both at Baseline and post-Baseline, the treatment group medians are imputed (based on the FAS).
2. If PAWP is missing either at Baseline or post-Baseline, the patient's available PAWP is imputed.

This imputation is based on the clinical assumption that macitentan does not affect PAWP.

Baseline: Patients without a Baseline PVR measurement is excluded from the analyses.

Post-Baseline: In patients with a post-Baseline PVR measurement obtained before Week 12, the last post-Baseline PVR measurement is carried forward.

If a patient has no post-Baseline PVR measurement, the ratio of Week 12 to Baseline PVR is imputed using the treatment group median based on the FAS.

Other imputation methods for PVR values are considered (e.g., multiple imputations) as sensitivity analysis.

### (i) Main Analysis

The primary analysis is performed on the FAS. PVR is summarized by time point and treatment group using descriptive statistics as well as geometric means and CVs. The ratio of Week 12 to Baseline PVR is summarized similarly.

The ratio of Week 12 to Baseline PVR is log-transformed (base e) and analyzed using an analysis of covariance (ANCOVA) with a factor for treatment group and a covariate for Baseline log PVR (macitentan vs. placebo). The treatment group difference (on log scale) and its 95% CLs are estimated based on the model. The geometric mean ratio (GMR; macitentan vs. placebo) and its 95% confidence interval are obtained by exponentiation. The null hypothesis is rejected if the entire 95% confidence interval is below one.

The treatment effect is expressed as (GMR-1)×100%, where a negative value indicates a reduction of PVR in the macitentan group as compared to the placebo group.

The log transformation for PVR is justified by the fact that ratios versus Baseline follow a normal distribution more closely after a log transformation. In addition, mean absolute changes from Baseline on log scale can be translated into (geometric) mean ratios by exponentiation.

### (ii) Supportive/Sensitivity Analyses

The primary and selected secondary efficacy endpoints are analyzed on the modified FAS as sensitivity analyses.

(iii) Analysis of the Secondary Efficacy Variables

Secondary efficacy analyses are performed on the FAS at α=0.025 (one-sided) using 95% CLs. No correction for multiple testing is applied for these analyses.

Changes from Baseline to Week 12 in pressure-volume variables (e.g., mRAP, mPAP, PAWP, CI TPR) and SVO₂ will be summarized and analyzed, but without the log-transformation.

NT-proBNP is summarized by time point and treatment group using descriptive statistics as well as geometric means and CVs. The ratio of Week 12 to Baseline NT-proBNP is summarized similarly. The ratio versus Baseline in NT-proBNP is log-transformed and analyzed using an ANCOVA with covariates for treatment group and Baseline log NT-proBNP.

WHO FC is summarized by time point and treatment group using frequency tables. Changes from Baseline in WHO FC are dichotomized as worsening (i.e., change >0) versus no change or improvement (i.e., change≤0). Worsening is analyzed using a logistic regression model with covariates for treatment group and WHO FC.

### (iv) Analysis of the Exploratory Efficacy Variables

Exploratory efficacy analyses are performed on the FAS at α=0.025 (one-sided) using 95% CLs.

Echocardiographic variables including TAPSE, S′, RVFAC, E′, A′, RVLS, RV area and RVSI are summarized by treatment and time point (Baseline, Week 12/EOT) using descriptive statistics (n, mean, SD, median, Q1, Q3, and range). The change from Baseline to Week 12/EOT in these variables is summarized similarly and analyzed using an ANCOVA with a factor for treatment and covariates Baseline log PVR and Baseline TAPSE, S′, RVFAC, E′, A′, RVLS, RV area and RVSI, respectively. The adjusted treatment effect and its 95% CI are presented.

Clinical events including: hospital admission for HF, re-initiation of i.v. diuretics ≥48 h, re-initiation of i.v. inotropes ≥48 h, need for RVAD/TAH, need for RRT and death are summarized by treatment group and time-to-event treatment differences are analyzed using the log-rank test.

Duration of hospital stay excluding rehabilitation stay are summarized by treatment group and analyzed using analysis of covariance with treatment and Baseline PVR in the model.

GFR is summarized by treatment and time point (Baseline, Week 12/EOT) using descriptive statistics (n, mean, SD, median, Q1, Q3, and range). The change from Baseline to Week 12/EOT in GFR is summarized similarly. Change from Baseline in GFR is analyzed using an ANCOVA with a factor for treatment and covariates Baseline log PVR and Baseline GFR. The adjusted treatment effect and its 95% confidence interval are presented.

### (vi) Analysis of the Safety Variables

The safety analyses described below are performed on the SS.

(vii) Adverse Events

A treatment-emergent AE is any AE temporally associated with the use of a treatment. The number and percentage of patients experiencing treatment-emergent AEs and SAEs at least once are tabulated by treatment group and by:
MedDRA SOC and individual preferred term within each SOC, in descending order of incidence.
Frequency of patients with events coded with the same preferred term, in descending order of incidence.

Furthermore, treatment-emergent AEs and SAEs are tabulated as described above by severity and relationship to treatment. AEs leading to premature discontinuation of treatment and death are also summarized as described above.

Listings are provided for all AEs, including SAEs. In addition, separate listings are provided for SAEs, for AEs leading to premature discontinuation of treatment, and for AEs leading to death.

### D. Laboratory Variables

Descriptive summary statistics by visit and treatment group are provided for observed values and absolute changes from Baseline, in both hematology and blood chemistry laboratory tests. In order to minimize missing data and to allow for Unscheduled Visits, all assessments up to EOT plus 30 days are assigned to the most appropriate visit time point according to the best fitting time-window for that assessment.

Laboratory variables are the following:
Hematology: hemoglobin, hematocrit, erythrocyte count, leukocyte count with differential counts, platelet count
Blood chemistry: AST, ALT, alkaline phosphatase, total and direct bilirubin, LDH, creatinine, BUN, uric acid, glucose, cholesterol, triglycerides, sodium, potassium, chloride, calcium, protein, albumin.

The number and percentage of patients with liver function test abnormalities are tabulated by treatment group. The number and percentage of patients with hemoglobin abnormalities are tabulated by treatment group.

Blood pressure (i.e., DBP and SBP), pulse rate, and body weight (in kg) are summarized at each visit using the usual location and scale summary statistics by treatment group for both absolute values and changes from Baseline. Patients for whom no post-Baseline value is available are excluded from the analysis of the changes from Baseline in the SS.

### Example 2: Administration of High Doses of Macitentan

This is a single-sequence, open-label, single-center, Phase 1 study in healthy male subjects. The study assessed the effect of 75 mg qd administration of macitentan. The study includes a screening phase of 21 days before the first macitentan administration, followed by an open-label treatment phase. Randomization was not used and subjects received the same treatment.

### A. Subject Population

29 subjects were enrolled. 11 subjects were enrolled under an initial protocol that did not include up-titration of macitentan treatment; 18 subjects were enrolled under an amended protocol that included up-titration of macitentan treatment.

### B. Study Treatment

B.1. Study Treatment without Uptitration of Macitentan

Administration of 75 mg qd macitentan started on Day 1. Macitentan was administered every day in the morning between 8:00 and 11:00 with 240 mL of noncarbonated water. One 75-mg macitentan film-coated tablet was thus administered as multiple qd oral doses under fed conditions.

Subjects received standardized meals and on dosing days when macitentan was administered in fed state, at the following time points:
Evening meal not later than 10 hours before macitentan administration next day.
Breakfast within 30 minutes prior to macitentan administration (Days 1-9 and Days 11-12).
Lunch approximately 4 hours after macitentan administration.
Snack approximately 7 hours after macitentan administration.
Evening meal approximately 10 hours after macitentan administration.

Subjects were planned to take macitentan 75 mg for 15 days. However the study was interrupted on the morning of day 8, i.e. when subjects had received 7 doses of macitentan 75 mg.

### B.2. Study Treatment Including Uptitration of Macitentan

Macitentan was administered in an up-titration regimen in the morning between 8:00 and 11:00 with 240 mL of noncarbonated water. One macitentan film-coated tablet (10 mg, 37.5 mg or 75 mg) was administered as multiple qd oral doses under fed conditions on Days 1-9 and Days 11-12, and under fasted conditions on Days 10 and 13. The up-titration regimen consists of 2 qd doses of 10 mg macitentan and 3 qd doses of 37.5 mg macitentan followed by 8 qd doses of 75 mg macitentan. On Days −4, −3, 10 and 13 (i.e., the days when dosing occurred under fasted conditions), no fluid intake apart from the water taken at the time of macitentan administration was allowed from 1 hour before until 1 hour after administration of macitentan.

Subjects received standardized meals and on dosing days when macitentan was administered in fed state, at the following time points:
Evening meal not later than 10 hours before macitentan administration next day.
Breakfast within 30 minutes prior to macitentan administration (Days 1-9 and Days 11-12).
Lunch approximately 4 hours after macitentan administration.
Snack approximately 7 hours after macitentan administration.
Evening meal approximately 10 hours after macitentan administration.

### C. Safety Measurements

Tables 18-20 summarize the frequency and timing of safety measurements.

TABLE 18
Screening Phase

| Study Day | Days −25 to −6 |
|---|---|
| Physical examination | X |
| Weight/Height (kg/cm) | X |
| Electrocardiogram (ECG) ^{b} | X |
| Vital signs ^{b} | X |
| Serology | X |
| eGFR^{f} | X |
| Hematology, chemistry, and urinalysis ^{d} | X |

TABLE 19
Treatment Phase (without up-titration)

| | Study Day | |
|---|---|---|
| | Day 1 | Day 2-9 |
| | Time | |
| | Predose | 0 |
| Physical examination | X | Day 9 |
| Symptom-directed physical examination | X | Days 2-9 |
| Weight (kg) | X | Days 2-9 |
| ECG ^{a} | X | Day 9 |
| Vital signs ^{a,b} | X | Days 3, 5, 7, 9 |
| Hematology, chemistry, and urinalysis ^{d} | X | Days 5, 9 ^{c} |
| Macitentan dosing ^{e} | X ^{c} | Days 2-9 |

| | |
|---|---|
| Adverse events | continuous |

^{a} Vital signs and ECGs measured after 5 minutes rest in supine position, the ECG using a standard 12-lead ECG. Blood pressure measured after approximately 2 minutes in standing position.
^{b} Vital signs include blood pressure (supine and standing), pulse rate, respiratory rate and temperature. Blood pressure and respiratory rate measured after 5 minutes rest in supine position. Standing blood pressure measured after approximately 2 minutes in standing position. Respiratory rate measured over at least 30 seconds.
^{c} Subjects fasted for at least 10 hours before dosing. Water (except for the 240 mL used for dosing) was restricted from 1 hour prior to until 1 hour after macitentan administration.
^{d} Subjects fasted for at least 10 hours.
^{e} Dosing under fed conditions.
^{f} eGFR was calculated according to the MDRD equation.

TABLE 20
Up-Titration Treatment Phase

| | Study Day | | | |
|---|---|---|---|---|
| | Day 1 | Day 2-9 | Day 10-12 | Day 13 |
| | Time | | | |
| | Predose | 0 | | |
| Physical examination | X | Day 9 | X | X |
| Weight | X | Days 2-9 | X | X |
| ECG ^{a,b} | X | Day 9 | X | X |
| Vital signs ^{a,b} | X | Days 3, | X | X |
| | | 5, 7, 9 | | |
| Drug/alcohol screen | X | | | |
| Hematology, chemistry, | X | Days 5 | X | X |
| and urinalysis ^{a,d} | | and 9 ^{e} | | |
| Macitentan dosing | X | X | X | X |

| | |
|---|---|
| Adverse events | continuous |

^{a} Vital signs and ECGs measured after 5 minutes rest in supine position, the ECG using a standard 12-lead ECG. Blood pressure measured after approximately 2 minutes in standing position.
^{b} Vital signs include blood pressure (supine and standing), pulse rate, respiratory rate and temperature. Blood pressure and respiratory rate measured after 5 minutes rest in supine position. Standing blood pressure measured after approximately 2 minutes in standing position. Respiratory rate measured over at least 30 seconds.
c. Subjects fasted for at least 10 hours before dosing. Water (except for the 240 mL used for dosing) was restricted from 1 hour prior to until 1 hour after macitentan administration.
^{d} Subjects fasted for at least 10 hours.
^{e} Dosing under fed conditions.
f. eGFR was calculated according to the MDRD equation.

The tests in Table 21 were performed:

TABLE 21
Hematology Panel

| | | |
|---|---|---|
| haemoglobin | platelet count | RBC count |
| haematocrit | differential blood count | differential |

Serum Chemistry Panel

| | | |
|---|---|---|
| sodium | Creatinine | lactic acid |
| | | dehydrogenase |
| potassium | Glucose | creatine |
| | | phosphokinase |
| chloride | AST | uric acid |
| calcium | ALT | albumin |
| bicarbonate | gamma- | total protein |
| | glutamyltransferase | |
| phosphate | total, indirect and | total cholesterol |
| | direct bilirubin | |
| urea | alkaline phosphatase | triglycerides |

Urinalysis

| | | |
|---|---|---|
| Dipstick | Ketones | epithelial cells |
| specific gravity | Bilirubin | crystals |
| pH | Urobilinogen | casts |
| glucose | Sediment | bacteria |
| protein | RBC | nitrite |
| blood | WBC | leukocyte esterase |

### D. Safety Analyses

All reported AEs with onset during the treatment phase were included in the analysis. The effects on cardiovascular variables were evaluated and the following variables were collected: PR (ms), QRS (ms), QT (ms), QTcB (ms), QTcF (ms), heart rate (bpm), and RR. The normal range for vital signs is defined as follows:
Systolic blood pressure: 100-140 mmHg
Diastolic blood pressure: 60-90 mmHg
Pulse rate: 45-90 bpm
Respiratory rate: 8-20 per minute
Body temperature: 35.0° C. to 37.5° C.

An AE is any untoward medical occurrence, i.e., any unfavorable and unintended sign (including an abnormal finding), symptom, or disease temporally associated with the use of macitentan. A treatment-emergent AE is any occurrence that is new in onset or aggravated in severity from the baseline condition. The following are used to assess all AEs:
Related: There is a reasonable causal relationship between macitentan administration and the AE.
Not Related: There is not a reasonable causal relationship between macitentan administration and the AE.

An assessment of AE severity grade was made using the following general categorical descriptors:
Mild: Awareness of symptoms that are easily tolerated, causing minimal discomfort and not interfering with everyday activities.
Moderate: Sufficient discomfort is present to cause interference with normal activity.
Severe: Extreme distress, causing significant impairment of functioning or incapacitation. Prevents normal everyday activities.

### E. Results

The AEs for subjects in the first cohort with and without up-titration were then analyzed. See, Table 22.

TABLE 21

| | | | | | Frequency |
|---|---|---|---|---|---|
| | | Severity | | Severity | observed |
| | | of AEs | | of AEs | with |
| | | (No | | (With | macitentan |
| | N | titration) | N | titration) | 10 mg |
| Total number of | 6 | | 6 | | |
| patients | | | | | |
| Headache | 6 | 5 mild | 6 | 5 mild | Very |
| | | 1 moderate | | 1 moderate | common |
| Sinusitis/rhinitis/ | 5 | All mild | 1 | Mild, less | Common |
| nasal congestion | | | | than 24 | |
| | | | | hours | |
| Flushing/feeling of | 4 | 3 mild | 0 | | Uncommon |
| warmth/redness of | | 1 moderate | | | |
| face and/or hands | | | | | |
| Photophobia | 3 | All mild | 0 | | Not known |

Frequencies are defined as: very common (≥1/10); common (≥1/100 to <1/10); uncommon (≥1/1,000 to <1/100); rare (≥1/10,000 to <1/1,000); very rare (<1/10,000); not known (cannot be estimated from the available data).

AEs were reported from Day 2 onwards, and were mainly mild. Four days after stopping macitentan most of the AEs resolved. For vital signs, ECG and lab parameters, no clinically significant changes reported. Further, a reduction in hemoglobin observed, within the expected range (No decrease >2 g/dL).

In summary, the data show that the introduction of an up-titration regimen improved tolerability of 75 mg macitentan. All subjects completed treatment with macitentan in the up-titration regimen cohort.

1. A method for treating pulmonary hypertension in a patient with left ventricular assist device (LVAD) implantation, comprising administering to a patient in need thereof a therapeutically effective amount of macitentan, wherein the LVAD implantation is within about 90 days prior to administering the macitentan.

2. The method of claim 1, wherein, after LVAD implantation and prior to initiating treatment with macitentan, the patient has a mean pulmonary arterial pressure (mPAP) of ≥25 mmHg at rest, a pulmonary arterial wedge pressure (PAWP)≤18 mmHg, and a pulmonary vascular resistance (PVR)>3 WU (Wood Units).

3. The method of claim 1, wherein the patient has a mean pulmonary arterial pressure (mPAP) of ≥25 mmHg at rest, a pulmonary arterial wedge pressure (PAWP)≤18 mmHg, and a pulmonary vascular resistance (PVR)>3 WU (Wood Units) within about 14 days prior to initiating the treatment with macitentan.

4. The method of claim 1, wherein the LVAD implantation is greater than 45 days to about 90 days prior to initiating the treatment with macitentan.

5. The method of claim 1, wherein the patient does not have Child-Pugh class C liver disease.

6. The method of claim 1, wherein the patient has a PVR of greater than 3 WU to about 7 WU after LVAD implantation and prior to initiating treatment with the macitentan.

7. The method of claim 1, wherein the method reduces PVR.

8. The method of claim 7, wherein the PVR is reduced by at least about 10% relative to a patient population at the same level of disease diagnosis that is not receiving treatment with macitentan.

9. The method of claim 1, wherein the patient has a transpulmonary gradient (TPG) of greater than about 12 mmHg to about 45 mmHg after LVAD implantation and prior to initiating treatment with macitentan.

10. The method of claim 9, wherein the method reduces the transpulmonary gradient.

11. The method of claim 10, wherein the TPG is reduced by at least about 10% relative to a patient population at the same level of disease diagnosis that is not receiving treatment with macitentan.

12. (canceled)

13. The method of claim 1, wherein the therapeutically effective amount of macitentan is about 10 mg.

14. The method of claim 1, wherein macitentan is administered orally in the form of a tablet once daily.

15. The method of claim 1, wherein the therapeutically effective amount of macitentan is about 60 mg to about 90 mg.

16. The method of claim 15, wherein the amount of macitentan is up-titrated from 10 mg per day, followed by from about 25 mg to about 50 mg per day, and followed by the therapeutically effective amount of about 60 mg to about 90 mg.

17. The method of claim 16, wherein the 10 mg per day of macitentan is administered for about 15 to about 45 days.

18. The method of claim 16, wherein the 25 mg to about 50 mg per day of macitentan is administered for about 15 to about 45 days.

19. The method of claim 15, wherein the therapeutically effective amount of macitentan is about 75 mg.

20. The method of claim 15, wherein macitentan is administered orally in the form of a tablet once daily.

21-29. (canceled)

30. A method of improving cardiac transplant eligibility in a patient with left ventricular assist device (LVAD) implantation, comprising administering to a patient in need thereof a therapeutically effective amount of macitentan, wherein the LVAD implantation is within 90 days prior to administering the macitentan.

31. The method of claim 30, wherein, after LVAD implantation and prior to initiating treatment with macitentan, the patient has a pulmonary vascular resistance (PVR)>3 WU (Wood Units).

32. The method of claim 30, wherein the patient has a pulmonary vascular resistance (PVR)>3 WU (Wood Units) within about 14 days prior to initiating the treatment with macitentan.

33. The method of claim 30, wherein the LVAD implantation is greater than 45 days to about 90 days prior to initiating the treatment with macitentan.

34. The method of claim 30, wherein the method reduces PVR of the patient below 3 WU.

35. (canceled)

36. The method of claim 30, wherein the therapeutically effective amount of macitentan is about 10 mg.

37. The method of claim 30, wherein macitentan is administered orally in the form of a tablet once daily.

38. The method of claim 30, wherein the therapeutically effective amount of macitentan is about 60 mg to about 90 mg.

39. The method of claim 38, wherein the amount of macitentan is up-titrated from 10 mg per day, followed by from about 25 mg to about 50 mg per day, and followed by the therapeutically effective amount of about 60 mg to about 90 mg.

40. The method of claim 39, wherein the 10 mg per day dose of macitentan is administered for about 15 day to about 45 days.

41. The method of claim 38, wherein the therapeutically effective amount of macitentan is about 75 mg.

42. The method of claim 38, wherein macitentan is administered orally in the form of a tablet once daily.

43. The method of claim 30, wherein the patient is on a cardiac transplant waiting list.

44. The method of claim 43, wherein the method results in the patient moving up on the waitlist or remaining on the waitlist.

45-110. (canceled)
